(19) United States
(12) Patent Application Publication
(10) Pub. No.: US 2021/0395684 A1
(43) Pub. Date: Dec. 23, 2021

(54) METHODS AND SYSTEMS FOR MANUFACTURING HEMATOPOIETIC LINEAGE CELLS

(71) Applicant: HebeCell Corporation, Natick, MA (US)

(72) Inventors: Qiang Feng, Natick, MA (US); Miao-yun Zhang, Natick, MA (US); Shi-jiang Lu, Natick, MA (US)

(21) Appl. No.: 17/287,281

(22) PCT Filed: Oct. 24, 2019
(86) PCT No.: PCT/US2019/057929

§ 371 Date: Apr. 21, 2021

Related U.S. Application Data
(60) Provisional application No. 62/749,947, filed on Oct. 24, 2018.

(51) Int. Cl. C12N 5/0789; C12N 5/0783; C12N 5/078

(52) U.S. Cl. CPC C12N 5/0647; C12N 5/0636; C12N 5/0641; C12N 2513/00; C12N 5/0646; C12N 2506/45; C12N 5/0644

(57) ABSTRACT

Provided herein, in one aspect, is hematopoietic lineage cells such as natural killer cells generated in vitro from human pluripotent stem cells (hPSCs) that can be used as a cell source for therapeutics. Methods and compositions for making and using the same are also provided.

### CROSS-REFERENCE TO RELATED APPLICATION

This application claims priority to and the benefit of U.S. Provisional Application No. 62/749,947 filed Oct. 24, 2018, the entire contents of which are incorporated herein by reference.

### FIELD

The present disclosure relates generally to methods and compositions for in vitro hematopoietic cell generation from, e.g., human pluripotent stem cells.

### BACKGROUND

Starting from very early stage of embryo development, hematopoiesis is a multistep process with the formation of all blood cell components. A healthy human adult must produce 10¹¹ to 10¹² blood cells per day to maintain normal body function. Transfusion of red blood cells (RBCs) and platelets saves life. Transplantation of hematopoietic stem cells (HSCs) from bone marrow, umbilical cord or peripheral blood are widely used clinically for many years for the treatment of blood malignancies and other disorders. More recently, other important hematopoietic cells such as dendritic cells (DC), T lymphocytes (T-Cells) and NK cells have been attracting enormous interest due to recent success in immuno-oncology.

Stem cells, particularly pluripotent stem cells (PSCs), can become any cell type in our body. Development of robust processes to manufacture high quality cells of desired lineage is the first step to fulfill the potential of this new technology. Lineage-specific differentiation of PSCs into mesodermal hematopoietic lineages has been extensively investigated (Ivanovs et al. 2017; Wahlster and Daley 2016). To achieve that, the following 4 different methodologies have been applied with various degrees of success. (1) Cytokine induction and co-culture with feeder cells (often derived from murine bone marrow stromal compartment) (Choi, Vodyanik, and Slukvin 2008); (2) Formation of embryoid bodies (EBs) and cytokine induction (Daley 2003; Lu et al. 2007); (3) Direct cytokine induction in 2D cultures (Feng et al. 2014; Salvagiotto et al. 2011); and (4) Forced induction by ectopic expression of lineage specific master transcription factors (Sugimura et al. 2017; Ebina and Rossi 2015).

Co-culture with bone marrow derived stromal cells has been a popular method for in vitro hematopoietic differentiation of PSCs. It has achieved better success at in vitro maturation of hematopoietic cells such as erythrocytes (Lu et al. 2008), megakaryocytes (Lu et al. 2011), and lymphocytes (Ditadi et al. 2015). However, the undefined nature of feeder cells of xeno origin as well as limited potential for scale up will make this method unsuitable for future therapeutic manufacture.

The EBs formation method, either through spontaneous or forced aggregation from PSC culture in a variety of formats is probably the most widely used method for lineage specific differentiation including hematopoietic differentiation. Spontaneous EBs formation is suitable only for small scale studies that do not require formation of EBs having uniform size. It therefore suffers from low differentiation efficiency and poor reproducibility. Forced aggregation of EBs using devices such as AggraWell (Stemcell Technology) can achieve EBs formation in desirable sizes (Ng et al. 2008). Such devices however are less likely to be adopted into system of large scale manufacture process. Additionally, multiple cases were observed in which specific PSC cell lines exhibited complete disintegration and significant cell death even after initial formation of EBs (unpublished data), suggesting large variations in cell lines for their capability to adapt from anchorage-dependent 2D to anchorage-independent 3D conditions.

Direct hematopoietic induction of 2D attached PSCs on specific matrix such as human collagen IV has been successfully established in recent years (Feng et al. 2014). However, it will require extremely large surface area to achieve large scale, commercially valuable production. Although theoretically possible with use of bioreactors having multi-layer flatbed culture surfaces, there are several technical and operational hurdles such as seeding PSCs at even density in such large area with tight space between layers, sampling, controlling of pH and gas exchange, feeding, and harvesting. All of these will inevitably lead to much higher cost for cell manufacture.

Thus, a need exists for a viable technology for manufacturing hematopoietic cells from PSCs at a scale suitable for therapeutic purpose.

### SUMMARY

The present disclosure provides, inter alia, a method for in vitro production of various hematopoietic lineage cells.

In one aspect, provided herein is a method for in vitro production of hematopoietic lineage cells, comprising:
(a) providing a plurality of first spheres comprising pluripotent stem cells (PSCs) in a first culture medium, wherein the first spheres have an average size of about 60-150 micrometers, about 70-120 micrometers or about 80-100 micrometers in diameter; wherein preferably the first spheres are generated from 3-dimensional (3D) sphere culturing while monitoring sphere size;
(b) 3D sphere culturing the plurality of first spheres in a second culture medium to induce differentiation of the PSCs to generate a plurality of second spheres comprising hemogenic endothelial cells (HECs);
(c) 3D sphere culturing the plurality of second spheres in a third culture medium to induce differentiation of the HECs to generate a plurality of third spheres comprising hematopoietic progenitor cells (HPCs);
(d) permitting the HPCs to release from the plurality of third spheres to obtain a suspension of substantially single cells of HPCs; and
(e) optionally, further differentiating the suspension of substantially single cells of HPCs into common erythroid/megakaryocytic progenitor cells, erythrocytes, megakaryocytes, platelets, common lymphoid progenitor cells, lymphoid lineage cells, lymphocytes (such as T lymphocytes), natural killer (NK) cells, common myeloid progenitor cells, common granulomonocytic progenitor cells, monocytes, macrophages, and/or dendritic cells.

In another aspect, a method for in vitro production of lymphoid lineage cells such as NK cells is provided, comprising:
(a) providing a plurality of first spheres comprising pluripotent stem cells (PSCs) in a first culture medium, wherein the first spheres have an average size of about 60-150 micrometers, about 70-120 micrometers or about 80-100 micrometers in diameter; wherein preferably the first spheres are generated from 3-dimensional (3D) sphere culturing while monitoring sphere size;
(b) 3D sphere culturing the plurality of first spheres in a second culture medium to induce differentiation of the PSCs to generate a plurality of second spheres containing hemogenic endothelial cells (HECs);
(c) enzymatically disassociating the plurality of second spheres to obtain a suspension of substantially single cells of HECs;
(d) seeding the substantially single cells of HECs into a scaffold that mimics in vivo hematopoietic niche; and
(e) culturing and differentiating, in the scaffold, the HECs into lymphoid lineage cells.

In a further aspect, a method for in vitro production of lymphoid lineage cells such as NK cells is provided, comprising:
(a) providing a plurality of first spheres comprising pluripotent stem cells (PSCs) in a first culture medium, wherein the first spheres have an average size of about 60-150 micrometers, about 70-120 micrometers or about 80-100 micrometers in diameter; wherein preferably the first spheres are generated from 3-dimensional (3D) sphere culturing while monitoring sphere size;
(b) 3D sphere culturing the plurality of first spheres in a second culture medium to induce differentiation of the PSCs to generate a plurality of second spheres containing hemogenic endothelial cells (HECs); and
(c) culturing and differentiating, in a scaffold-free third culture medium, the HECs in the second spheres into lymphoid lineage cells, while permitting the lymphoid lineage cells to release from the second spheres.

In various embodiments, the PCSs used in the method disclosed herein can be embryonic stem cells or induced pluripotent stem cells, preferably from human. In some embodiments, the PCSs are at least 95% positive for Oct-4 expression.

In some embodiments, 3D sphere culturing comprises culturing in a spinner flask or stir-tank bioreactor, preferably under continuous agitation.

In certain embodiments, the first culture medium is a PSC culture medium supplemented with TGF-β of about 1-10 ng/mL, bFGF of about 10-500 ng/mL, and Y27632 of about 1-5 μM. In some embodiments, the PSC culture medium is NutriStem®, mTeSR™1, mTeSR™2, TeSR™-E8™ or other culture medium suitable for 3D suspension culture.

In some embodiments, the second culture medium is a PSC culture medium supplemented with BMP4, VEGF and bFGF, each preferably at a concentration of about 25 to about 50 ng/mL, and optionally supplemented with CHIR99012 and/or SB431542, each preferably at a concentration of about 1-10, about 2-5, or about 3 μM. In some embodiments, the PSC culture medium is NutriStem®, mTeSR™1, mTeSR™2, TeSR™-E8™ or other culture medium suitable for 3D suspension culture. In some embodiments, the second culture medium can be supplemented with (i) BMP4, VEGF and bFGF for a first period of time (e.g., day 1 and day 2), (ii) BMP4, VEGF, bFGF and CHIR99012 for a second period of time (e.g., day 3), (iii) BMP4, VEGF, bFGF, CHIR99012 and SB431542 for a third period of time (e.g., day 4), (iv) BMP4, VEGF, bFGF, and SB431542 for a fourth period of time (e.g., day 5), and (v) BMP4, VEGF and bFGF for a fifth period of time (e.g., day 6). In some embodiments, said culturing in the second culture medium is under hypoxia condition (about 5% oxygen) for the first period of time through the third period of time (e.g., day 1 through day 4), followed by normal oxygen concentration of about 20% for the fourth period of time and the fifth period of time (e.g., day 5 and day 6).

In some embodiments, the third culture medium is a hematopoietic basal medium supplemented with one or more of TPO, SCF, Flt3L, IL-3, IL-6, IL-7, IL-15, SR1, sDLL-1, OSM and/or EPO. In some embodiments, the hematopoietic basal medium is StemSpan™-ACF, PRIME-XV®, PromoCell® Hematopoietic Progenitor Expansion medium DXF and other culture system suitable for hematopoietic stem cell expansion.

In some embodiments, step (e) can comprise culturing in a hematopoietic basal medium supplemented with one or more of TPO, SCF, Flt3L, IL-3, IL-6, IL-7, IL-15, SR1, sDLL-1, OSM and/or EPO. In some embodiments, the hematopoietic basal medium is StemSpan™-ACF, PRIME-XV®, PromoCell® Hematopoietic Progenitor Expansion medium DXF and other culture medium suitable for lineage-specific expansion and maturation.

Also provided herein is a method of treating cancer and other immune diseases, comprising: administering to a patient in need thereof a plurality of cells produced using any one of the methods disclosed herein. In some embodiments, the cells have been engineered to express a chimeric antigen receptor, a T-cell receptor or other receptor for disease antigens. In some embodiments, the cells are lymphoid lineage cells such as T-cells, NK cells, dendritic cells and/or macrophages.

A composition for adoptive cell therapy is also provided, which can comprise a plurality of cells produced using any one of the methods disclosed herein. In some embodiments, the cells have been engineered to express a chimeric antigen receptor, a T-cell receptor or other receptor for disease antigens for the treatment of cancer or other immune diseases. In some embodiments, the cells are lymphoid lineage cells such as T-cells, NK cells, dendritic cells and/or macrophages.

A further aspect relates to cells produced using any one of the methods disclosed herein for the treatment of cancer or other immune diseases. In some embodiments, the cells have been engineered to express a chimeric antigen receptor, a T-cell receptor or other receptor for disease antigens. In some embodiments, the cells are lymphoid lineage cells such as T-cells, NK cells, dendritic cells and/or macrophages.

Also provided are the culture medium compositions disclosed herein.

### BRIEF DESCRIPTION OF THE DRAWINGS

FIGS. 1A-1E illustrate morphology and characterization of pluripotent stem cells suitable for 3D hematopoietic differentiation. FIG. 1A is an image of a typical spinner flask style bioreactor. FIG. 1B is an image of PSC cell spheres in low magnification (40×). FIG. 1C is an image of PSC cell spheres in high magnification (100×, scale bar=200 uM). FIG. 1D is a graph depicting representative flow cytometer results of Oct-4 expression in undifferentiated PSCs. FIG. 1E provides a representative karyotyping showing a normal female karyotype.

FIG. 2 is a schematic description of stepwise hematopoietic induction process under 3D sphere condition.

FIGS. 3A-3C characterize an HEC population during first 6 days of differentiation. FIG. 3A is a graph depicting impact of starting sphere size on HEC differentiation efficiency. FIG. 3B is flow cytometry data depicting representative of time-dependent expression of HEC markers CD31, CD144, CD34, hematopoietic markers CD43, CD41, CD235a and CD45, and loss of pluripotency marker of Oct-4. FIG. 3C is a graph depicting quantitative profiling of HE related surface marker expression from HEC on day 6 of differentiation.

FIGS. 4A-4I depict stage-dependent morphology of cell spheres. FIG. 4A is an image of sphere morphology of undifferentiated PSCs. FIG. 4B is an image of day 3 spheres. FIG. 4C is an image of day 6 spheres. FIG. 4D is an image of day 9 spheres. FIG. 4E is an image of day 12 spheres. FIG. 4F is an image of day 15 spheres. FIG. 4G is an image of day 15 spheres in 100× magnification showing released HPCs between large spheres. FIG. 4H is an image of day 19 spheres. FIG. 4I is an image of day 22 spheres. All images are at 40× magnification unless stated otherwise.

FIG. 5 comprises images depicting histology and immunofluorescence of HEC lineage specific marker expression in spheres at different stage of differentiation. Top row: cross sections of spheres at Day 0, 6, 9, 14 and 23 of differentiation; Second row: HEC marker CD31(green) expression in the cross section of spheres at Day 0, 6, 9, 14 and 23 of differentiation, cell nuclear were stained with DAPi (blue); Third row: HEC marker CD34 (green) expression in the cross section of spheres at Day 0, 6, 9, 14 and 23 of differentiation, cell nuclear were stained with DAPi (blue); Bottom row: hematopoietic marker CD43 (green) expression in the cross section of spheres at Day 0, 6, 9, 14 and 23 of differentiation, cell nuclear were stained with DAPi (blue). All images are at 100× magnification.

FIGS. 6A-6E illustrate time-dependent lineage-specific marker expression in dissociated sphere cells. FIG. 6A is a graph depicting flow cytometer analysis of CD31 in cell spheres from experiments Cond. A and Cond. B. FIG. 6B is a graph depicting flow cytometer analysis of CD34 in cell spheres from experiments Cond. A and Cond. B. FIG. 6C is a graph depicting flow cytometer analysis of CD43 in cell spheres from experiments Cond. A and Cond. B. FIG. 6D is a graph depicting flow cytometer analysis of CD235a in cell spheres from experiments Cond. A and Cond. B. FIG. 6E is a graph depicting flow cytometer analysis of CD45 in cell spheres from experiments Cond. A and Cond. B

FIGS. 7A and 7B depict quantity of time-dependent released of HPCs from 3D cultured spheres. FIG. 7A is a table depicting the number of daily harvests of HPCs from experiment Cond. A and Cond. B from day 9 until Day 23. FIG. 7B is a graph illustrating the HPCs harvest number from both conditions.

FIGS. 8A-8E depict hematopoietic lineage-specific marker expression in harvested HPCs released from 3D spheres. FIG. 8A comprises representative flow cytometer analysis of HPC harvested on day 9, for paired marker expression profile from left to right: CD31/CD43; CD34/CD45; CD34/CD133; CD41/CD235a; CD45/CD235a and CD41/CD45. FIG. 7B is a graph showing CD31, CD43 single and combined expression profile of HPCs harvested on different days of sphere differentiation. FIG. 7C is a graph showing CD34 and CD45 single or combined expression profile of HPCs harvested on different days of sphere differentiation. FIG. 7D is a graph showing CD41, CD235a and CD45 expression profile of HPCs harvested on different days of sphere differentiation. FIG. 7E is a graph showing CD41/CD235a, CD45/CD235a and CD41/CD45 combined expression profile of HPCs harvest on different days of sphere differentiation.

FIGS. 9A-9L illustrate CFU forming capability of CD34⁺ cells purified from dissociated spheres on day 22 or differentiation. FIG. 9A is a whole culture view of CFU forming capability of CD34⁺ (left), CD34⁻CD45⁺ (center) and CD34⁻CD45⁻ cells. FIG. 9B is a graph depicting the number and phenotypes of Colony Forming Units (CFUs) from CD34⁺, CD34⁻CD45⁺ and CD34⁻CD45⁻ cells. FIG. 7C is flow cytometry data showing the expression of CD133 in CD34⁺ cells. FIG. 9D is an image of a large burst BFU-E. FIG. 9E is an image of a large CFU-E. FIG. 9F is an image of CFU-E and CFU-M. FIG. 9G is an image of a large red CFU-mix colony. FIG. 9H is an image of a small CFU-E. FIG. 9I is an image of a red CFU-mix. FIG. 9J is an image of a CFU-G. FIG. 9K is an image of CFU-M and -G. FIG. 9L is an image of CFU-M. All micrograph images are at 40× magnification.

FIGS. 10A-10E depict HPCs released from 3D spheres had both megakaryocytic and erythroid potentials. FIG. 10A comprises microscope images of HPC-derived megakaryocytes in MK-specific cultures showing extensive pro-platelet formation (indicated by arrows). FIG. 10B is a forward and side scatter plot for MK (P2) and platelet (P1) rich population. FIG. 10C is flow cytometry data showing that MKs in gate P2 are 83.4% CD41⁺CD42⁺. FIG. 10D is flow cytometry data of platelets in P1 showing 66.2% CD41⁺CD42⁺. FIG. 10E comprises images of the morphology of large expanded red blood cell colonies derived from HPCs released from spheres on day 10 (40× magnification).

FIGS. 11A-11D depict the derivation of CD56^{+high} NK cells from early HPCs released on day 8. FIG. 11A is a graph characterizing HPCs (HPC-A: day 8; HPC-B: day 11; HPC-C: day 18) prior to initiating NK differentiation. FIG. 11B comprises flow cytometry data that characterizes CD56^{+high} NK cells in NK differentiation cultures. FIG. 11C is a graph depicting time-dependent CD56 expression of NK differentiation in medium #1. FIG. 11D is a graph depicting time-dependent CD56 expression of NK differentiation in medium #2.

FIGS. 12A-12D characterize iPS-NK cells in vitro. FIG. 12A is an image of typical HPC morphology (400× magnification). FIG. 12B is an image showing the morphology of iPS-NK cells released on day 30 (400× magnification). FIG. 12C comprises forward and side scattering plots of: iPS-NK cells (top left); TCR expression on CD56⁺ iPS-NK cells (top middle); PBMC positive control for TCR antibody (top right); CD3 expression in CD56⁺ iPS-NK cells (Lower left); PBMC positive control for CD3 antibody (lower middle), CD19 expression in iPS-NK cells (lower right). FIG. 12D comprises forward and side scattering plots of: CD56⁺ iPS-NK cells are NKG2D⁺ (top left), NKp44⁺ (middle left); and NKp46+(lower left); 49.2% CD56⁺ iPS-NK cells are KIR2DS4⁺ (top right), 31.8% CD56⁺ iPS-NK cells are KIR2DL1/DS1⁺ (middle right); CD56⁺ iPS-NK cells are KIR3DL1/DS1⁻ (lower right).

FIG. 13 comprises flow cytometry data showing cytotoxic activity of iPS-NK cells on K562 target cells. Top row: K562 cells only control; Second row: E:T ratio at 12.5:1; Third row: E:T ratio at 25:1; bottom row: E:T ratio at 50:1. Left column: forward and side scattering profiles of target cells (P1) and effector cells (P2); Second column from left: GFP histogram of both K562 (positive) and NK (negative); Second column from right: percentage of dead cells in GFP+K562 (gate M2).

FIG. 14 shows that over 80% of human iPS-NK cells are CD56+CD8+ effector cells. Panel A is flow cytometry data showing CD56+ human iPS-NK cells do not express CD3. Panel B is flow cytometry data showing 80% of CD56+ iPS-NK cells express CD8 antigen, but not CD4 antigen. Panel C is flow cytometry data showing that >80% of CD56+ iPS-NK cells from a different batch express CD8 antigen, but not CD3 antigens. Panel D is flow cytometry data showing that >80% of CD56+ iPS-NK cells from a different batch express CD8 antigen, but not TCR antigens.

FIGS. 15A-15D depict human iPS-NK cells expansion under feeder-free conditions. Total of 5 different batches of harvested iPS-NK cells/progenitors were expanded in vitro using our newly developed feeder-free defined medium. FIG. 15A is a graph showing between 2.4 and 5.6-fold increase in cell numbers were achieved with average fold increase of 3.83. FIG. 15B is a graph showing significant enrichment of NK population was achieved, from average 37.8% of CD56+ cells pre-expansion to average 95.2% of CD56+ cells post expansion, with highest purity reached 99%. FIG. 15C comprises flow cytometry data that illustrates representative co-expression of CD56/NKG2D, CD56/NKP44 and CD56/NKP46 in pre-expanded iPS-NK cells. FIG. 15D comprises flow cytometry data that illustrates representative co-expression of CD56/NKG2D, CD56/NKP44 and CD56/NKP46 in post-expanded iPS-NK cells.

FIGS. 16A-16G illustrate NK cell lineage specific differentiation in a 500-ml bioreactor. FIG. 16A is a graph showing efficient induction of hemogenic endothelial markers CD31, CD144, CD34 in day 3 and day 5 spheres from both 30 ml and 500 ml bioreactors. Induction of early hematopoietic marker CD43 in day 3 and day 5 spheres are also comparable; FIG. 16B is a graph illustrating the expression of NK marker CD56 in harvested cells from a 500-ml bioreactor (red line) demonstrated a very similar pattern with cells harvested from 3 individual 30 ml bioreactors. FIG. 16C is an image showing iPS-NK cells harvested from 500 ml bioreactors showed homogenous NK cell morphology. FIG. 16D is flow cytometry data showing that over 90% iPS-NK cells harvested from 500 ml are CD56+, and these cells also express NKG2D. FIG. 16E is flow cytometry data showing that over 90% iPS-NK cells harvested from 500 ml are CD56+, and these cells also express NKp46. FIG. 16F is flow cytometry data showing that over 90% iPS-NK cells harvested from 500 ml are CD56+, and these cells also express NKp44. FIG. 16G is flow cytometry data showing that over 90% iPS-NK cells harvested from 500 ml are CD56+, and these cells also express and KIR.

FIG. 17 shows CD3+T lymphocytes generated from the presently disclosed 3D hematopoietic differentiation platform. Expression of T cell marker CD3 and NK cell marker CD56/NKG2D in cells harvested from two individual bioreactors are shown. Panels A and C: 64.5% and 61.7% cells harvested from bioreactor #1 and #2 are CD3⁺CD8⁻, respectively. Panels B and D: 61.5% and 77% of cells harvested from bioreactor #1 and #2 are CD56⁻NKG2D⁻, respectively.

FIG. 18 illustrates that human iPS-NK selectively kill K562 cancer cells but not normal cells. Green fluorescence labelled K562 cancer cells or normal human peripheral mononucleotide cells (PBMC) were mixed with human iPS-NK cells at 1:1 ratio and cytotoxic effect were measured by flow cytometer after 2 hours incubation. Panel A: iPS-NK cells before mixing with PBMC. Panel B: PBMC before mixing with iPS-NK cells. Panel C: iPS-NK cells and PBMC 2 hours after mixing with each other, PBMC remained intact. Panel D: iPS-NK cells before mixing with K562 cells. Panel E: K562 cells before mixing with iPS-NK cells. Panel F: iPS-NK cells and K562 cells 2 hours after mixing with each other, >80% of K562 cells were killed.

### DETAILED DESCRIPTION

Provided herein, in one aspect, is a novel methodology suitable for manufacturing hematopoietic cells at industrial scale to meet the demand for cell therapies. Starting with PSCs in 3D culture, these cells can first be differentiated into hemogenic endothelial cells (HEC), which are intermediate population with bi-potentials to become both hematopoietic as well as endothelial cell lineages (Ditadi et al. 2015; Feng et al. 2014; Swiers et al. 2013). After transition to conditions suitable for hematopoietic commitment and expansion, significant number of hematopoietic progenitor cells (HPC) can be released from the 3D spheres. Progenitors at various stages of expansion can be harvested, analyzed for their phenotype and function, and cryopreserved. The whole manufacturing process is developed under 3D suspension culture condition which can be easily adapted into commercially available single-use stir tank bioreactors or other large-scale cell manufacturing devices. The method disclosed herein is highly efficient and reproducible with easy access to cell sampling and harvesting at any time during the process. The system can be customized for manufacturing cells of various differentiation stages and different lineages such as HECs, hematopoietic stem/progenitor cells, erythroid/megakaryocytic progenitors, myeloid progenitors, lymphoid progenitors, as well as matured erythrocytes, platelets, T lymphocytes, and NK cells.

In some embodiments, a highly reproducible and scalable cell manufacture platform technology is provided that is capable of efficiently converting human PSC spheres, in a well-controlled stepwise fashion, firstly into spheres containing a high percentage of HECs. The HEC-rich spheres can be subsequently transitioned into spheres containing high activity of hematopoiesis that can release large quantity of HPCs with all hematopoietic lineage potentials. These HPCs can robustly differentiate into all hematopoietic lineage cells including, but not limited to, megakaryocytes/platelets and natural killer (NK) cells. In some embodiments, such NK cells derived from human PSCs can be utilized as off-the-shelf products for cancer immunotherapy.

Significantly, using the method disclosed herein, the cells are processed under defined 3D suspension culture conditions without any feeder cells or carriers, which can be easily adopted into various forms of single-use bioreactors. Secondly, the 3D culture method and system disclosed herein can be used to manufacture a variety of hematopoietic cells. Thirdly, the 3D culture method and system disclosed herein is process friendly as HPCs are naturally released from spheres, which allows cell harvesting with high viability and functionality.

In some embodiments, the 3D culture method and system disclosed herein is estimated to have an input to output ratio (PSC:HPC) of at least 1:5, 1:10, at least 1:20, at least 1:30, or about 1:31. For example, a 1:31 PSC:HPC ratio allows the manufacture of up to 5.6×10¹⁰ HPCs from a single bioreactor with a working volume of 3 liters. The simplicity of this platform provides a solid foundation for any system modifications required for manufacturing cells of different lineages. The scalability of this 3D platform also makes it a desirable option to manufacture large scale of cells for both autologous and allogeneic therapies.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The articles “a” and “an” are used herein to refer to one or to more than one (i.e., at least one) of the grammatical object of the article. By way of example, “an element” means one element or more than one element.

As used herein, the term “about” means within 20%, more preferably within 10% and most preferably within 5%. The term “substantially” means more than 50%, preferably more than 80%, and most preferably more than 90% or 95%.

As used herein, “a plurality of” means more than 1, e.g., 2, 3, 4, 5, 6, 7, 8, 9, 10, 11, 12, 13, 14, 15, 16, 17, 18, 19, 20, or more, e.g., 25, 30, 40, 50, 60, 70, 80, 90, 100, 200, 300, 400, 500, or more, or any integer there between.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The term “embryonic stem cells” (ES cells or ESCs) refers to pluripotent cells derived from the inner cell mass of blastocysts or morulae that have been serially passaged as cell lines. The ES cells may be derived from fertilization of an egg cell with sperm or DNA, nuclear transfer, parthenogenesis etc. The term “human embryonic stem cells” (hES cells) refers to human ES cells. The generation of ESC is disclosed in U.S. Pat. Nos. 5,843,780; 6,200,806, and ESC obtained from the inner cell mass of blastocysts derived from somatic cell nuclear transfer are described in U.S. Pat. Nos. 5,945,577; 5,994,619; 6,235,970, which are incorporated herein in their entirety by reference. The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Such cells include hES cells, human embryo-derived cells (hEDCs), and adult-derived stem cells. Pluripotent stem cells may be genetically modified. In some embodiments, the pluripotent stem cells are not genetically modified. Genetically modified cells may include markers such as fluorescent proteins to facilitate their identification. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refers to a pluripotent stem cell artificially derived (e.g., induced or by complete reversal) from a non-pluripotent cell, typically an adult somatic cell, for example, by inducing a forced expression of one or more genes.

The term “reprogramming” as used herein refers to the process that alters or reverses the differentiation state of a somatic cell, such that the developmental clock of a nucleus is reset; for example, resetting the developmental state of an adult differentiated cell nucleus so that it can carry out the genetic program of an early embryonic cell nucleus, making all the proteins required for embryonic development. Reprogramming as disclosed herein encompasses complete reversion of the differentiation state of a somatic cell to a pluripotent or totipotent cell. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The terms “renewal” or “self-renewal” or “proliferation” are used interchangeably herein, are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “culture” or “culturing” as used herein refers to in vitro laboratory procedures for maintaining cell viability and/or proliferation.

The term “carrier-free three-dimension sphere” culture or culturing refers to a technique of culturing the cells in nonadherent conditions such that the cells can form spheres by themselves without any carriers. A conventional method for culturing cells having adhesiveness is characterized in that cells are cultured on a plane of a vessel such as a petri dish (two-dimensional culture). In contrast, in the three-dimensional cultivation method, no adherence cue is provided to the cells and the culture is largely dependent on cell-cell contacts. As used herein, “carriers” or “microcarriers” refer to solid spherical beads made with plastic, ceramics or other materials such as gelatin or hydrogel, designed to provide adherent surface for suspension cell culture. Carrier with other form or shape have also been reported such as fibrous structure.

The term “scaffold” refers to solid or semi-solid materials that have been engineered to cause desirable cellular interaction to contribute to the formation of new functional tissues for tissue engineering and regeneration. In some embodiments, cells are often seeded into these structures capable of supporting three-dimensional tissue formation. Scaffolds mimic the extracellular matrix of the native tissue, recapitulate the in vivo milieu and allow cells to influence their own microenvironments. They usually serve at least one of the following purposes: allow cell attachment and migration, deliver and retain cells and biomedical factors, enable diffusion of vital cell nutrients and expressed products, exert certain mechanical and biological influences to modify behaviors of cells. To achieve the goal of tissue reconstruction, scaffolds must meet certain specific requirements. A high porosity and adequate pore size are necessary to facilitate cell seeding and diffusion. Scaffold materials must be biocompatible. In some embodiments, biodegradable materials were used. In some embodiments, the scaffolds can be dissolved by enzymatic treatment, or by change of physical conditions such as pH and/or temperature etc. to facilitate recovery or harvest of cells within scaffolds. In some embodiments, porous scaffolds can also be used as carriers for optimal cell differentiation and manufacture. The physical characterization of scaffolds such as pore size, rigidity, content of extracellular matrix and shape can be customized for optimal growth of tissues, such as, but not limited to, bone, heart, liver, and dermal tissues. In some embodiments, the scaffold can be selected to mimic the in vivo niche to promote lineage specification such as NK cells, T lymphocytes, etc.

The term “sphere” or “spheroid” means a three-dimensional spherical or substantially spherical cell agglomerate or aggregate. In some embodiments, extracellular matrices can be used to help the cells to move within their spheroid similar to the way cells would move in living tissue. The most common types of ECM used are basement membrane extract or collagen. In some embodiments, a matrix- or scaffold-free spheroid culture can also be used, where cells are growing suspended in media. This could be achieved either by continuous spinning or by using low-adherence plates. In embodiments, spheres can be created from single culture or co-culture techniques such as hanging drop, rotating culture, forced-floating, agitation, or concave plate methods (see, e.g., Breslin et al., Drug Discovery Today 2013, 18, 240-249; Pampaloni et al., Nat. Rev. Mol. Cell Biol. 2007, 8, 839-845; Hsiao et al., Biotechnol. Bioeng. 2012, 109, 1293-1304; and Castaneda et al., J. Cancer Res. Clin. Oncol. 2000, 126, 305-310; all incorporated by reference). In some embodiments, the size of the spheres can grow during 3D culturing.

As used herein, “feeder-free” refers to a condition where the referenced composition contains no added feeder cells. As used herein, “feeder cells” refers to non-PS cells that are co-cultured with PS cells and provide support for the PS cells. Support may include facilitating the growth and maintenance of the PS cell culture by producing one or more growth factors. Example of feeder cells may include cells having the phenotype of connective tissue such as murine fibroblast cells, human fibroblasts.

The term “culture medium” is used interchangeably with “medium” and refers to any medium that allows cell proliferation. The suitable medium need not promote maximum proliferation, only measurable cell proliferation. In some embodiments, the culture medium maintains the cells in a pluripotent state. In some embodiments, the culture medium encourages the cells (e.g., pluripotent cells) to differentiate into, e.g., HECs and HPCs. A few exemplary basal media used herein include DMEM/F-12 (Dulbecco's Modified Eagle Medium/Nutrient Mixture F-12; available from Thermo Fisher Scientific), Growth Factor-Free NutriStem® Medium which contains no bFGF or TGFβ (GF-free NutriStem®, available from Biological Industries), NutriStem® hPSC XF Medium® (Biological Industries), mTeSR™1 (STEMCELL Technologies Inc.), mTeSR™2 (STEMCELL Technologies Inc.), TeSR™-E8™ (STEMCELL Technologies Inc.), StemSpan™-ACF (STEMCELL Technologies Inc.), PRIME-XV®(Irving Scientific), and PromoCell® Hematopoietic Progenitor Expansion medium DXF (PromoCell GmbH). Each can be supplemented with one or more of: suitable buffer (e.g., HEPES (4-(2-hydroxyethyl)-1-piperazineethanesulfonic acid)), chemically-defined supplements such as N2 (0.1-10%, e.g., 1%) and B27 (0.1-10%, e.g., 1%) serum-free supplements (available from Thermo Fisher Scientific), antibiotics such as penicillin/streptomycin (0.1-10%, e.g., 1%), MEM non-essential amino acids (Eagle's minimum essential medium (MEM) which is composed of balanced salt solutions, amino acids and vitamins that are essential for the growth of cultured cells, which, when supplemented with non-essential amino acids, makes MEM non-essential amino acid solution), glucose (0.1-10%, e.g., 0.30%), L-glutamine (e.g., GlutaMAX™), ascorbic acid, and/or DAPT (N-[N-(3,5-difluorophenacetyl)-1-alanyl]-S-phenylglycine t-butyl ester). Factors for inducing differentiation such as Heparin, bone morphogenetic protein 4 (BMP4), oncostatin M (OSM), vascular endothelial growth factor (VEGF), basic fibroblast growth factor (bFGF), thrombopoietin (TPO), stem cell factor (SCF), soluble delta-like protein 1 (sDLL-1), erythropoietin (EPO), FMS-like tyrosine kinase 3 ligand (Flt3L), interleukin (IL)-3, IL-6, IL-9, IL-7, IL-15, Y27632, CHIR99021, SB431542, and/or StemRegenin 1 (SR1) as disclosed herein can also be added to the medium.

The term “differentiated cell” as used herein refers to any cell in the process of differentiating into a somatic cell lineage or having terminally differentiated. In the context of cell ontogeny, the adjectives “differentiated” and “differentiating” are relative terms meaning a “differentiated cell” that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as a hematopoietic progenitors), and then to an end-stage differentiated cell, which plays a characteristic role in a certain tissue type, and may or may not retain the capacity to proliferate further.

The terms “enriching” and “enriched” are used interchangeably herein and mean that the yield (fraction) of cells of one type is increased by at least 10% over the fraction of cells of that type in the starting culture or preparation.

The term “agent” as used herein means any compound or substance such as, but not limited to, a small molecule, nucleic acid, polypeptide, peptide, drug, ion, etc. An agent can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The term “small molecule” refers to an organic compound having multiple carbon-carbon bonds and a molecular weight of less than 1500 daltons. Typically, such compounds comprise one or more functional groups that mediate structural interactions with proteins, e.g., hydrogen bonding, and typically include at least an amine, carbonyl, hydroxyl or carboxyl group, and in some embodiments at least two of the functional chemical groups. The small molecule agents may comprise cyclic carbon or heterocyclic structures and/or aromatic or polyaromatic structures substituted with one or more chemical functional groups and/or heteroatoms.

The term “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, the ability to incorporate or exclude particular dyes, the ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified,” with regard to a population of definitive endoderm cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not definitive endoderm cells or their progeny as defined by the terms herein. In some embodiments, the present disclosure encompasses methods to expand a population of definitive endoderm cells, wherein the expanded population of definitive endoderm cells is a substantially pure population of definitive endoderm cells. Similarly, with regard to a “substantially pure” or “essentially purified” population of pluripotent stem cells, refers to a population of cells that contain fewer than about 20%, more preferably fewer than about 15%, 10%, 8%, 7%, most preferably fewer than about 5%, 4%, 3%, 2%, 1%.

“Hematopoietic lineage cells,” as used herein, refers to cells differentiated in vitro from PSCs and/or their progeny and may include one or more of the following: hemangioblasts, hemogenic endothelial cells (HECs), hematopoietic stem cells, hematopoietic progenitor cells (HPCs), erythroid/megakaryocytic progenitor cells, erythrocytes, megakaryocytes, platelets, and lymphoid lineage cells. The term “lymphoid lineage cells” includes one or more of: lymphoid progenitor cells, lymphocytes (such as T lymphocytes), natural killer (NK) cells, myeloid progenitor cells, granulomonocytic progenitor cells, monocytes, macrophages, and dendritic cells.

“Hemogenic endothelial cells” refers to cells differentiated in vitro from PSCs that acquire hematopoietic potential and can give rise to multilineage hematopoietic stem and progenitor cells. Human markers for HECs include CD31, CD144, CD34, and CD184.

“Hematopoietic progenitor cell” refers to a cell that remains mitotic and can produce more progenitor cells or precursor cells or can differentiate to an end fate hematopoietic cell lineage. Human markers for HPCs include: CD31, CD34, CD43, CD133, CD235a, CD41 and CD45, wherein CD41+ indicates megakaryocyte progenitors, CD235a+ erythrocyte progenitors, CD34⁺CD45⁺ early lymphoid/myeloid lineage progenitors, CD56⁺ NK lineage progenitors, and CD34⁺CD133⁺ hematopoietic stem cells.

The term “treatment” or “treating” means administration of a substance for purposes including: (i) preventing the disease or condition, that is, causing the clinical symptoms of the disease or condition not to develop; (ii) inhibiting the disease or condition, that is, arresting the development of clinical symptoms; and/or (iii) relieving the disease or condition, that is, causing the regression of clinical symptoms.

As used herein, the term “cancer” refers to or describes the physiological condition in mammals that is typically characterized by unregulated cell growth. Examples of cancer include, but are not limited to, melanoma, carcinoma, lymphoma, blastoma, sarcoma, and leukemia or lymphoid malignancies. More particular examples of cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The term “disease antigen” as used herein refers to a macromolecule, including all proteins or peptides that are associated with a disease. In some embodiments, an antigen is a molecule that can provoke an immune response, e.g., involving activation of certain immune cells and/or antibody generation. T cell receptors also recognized antigens (albeit antigens whose peptides or peptide fragments are complexed with an MHC molecule). Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full-length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response, including tumor-associated antigens.

“Tumor-associated antigen” (TAA) is an antigenic substance produced in tumor cells that triggers an immune response in the host. Tumor antigens are useful tumor markers in identifying tumor cells with diagnostic tests and are potential candidates for use in cancer therapy. In some embodiments, the TAA can be derived from, a cancer including but not limited to primary or metastatic melanoma, thymoma, lymphoma, sarcoma, lung cancer, liver cancer, non-Hodgkin's lymphoma, non-Hodgkins lymphoma, leukemias, uterine cancer, cervical cancer, bladder cancer, kidney cancer and adenocarcinomas such as breast cancer, prostate cancer, ovarian cancer, pancreatic cancer, and the like. TAAs can be patient specific. In some embodiments, TAAs may be p53, Ras, beta-Catenin, CDK4, alpha-Actinin-4, Tyrosinase, TRP1/gp75, TRP2, gplOO, Melan-A/MART 1, Gangliosides, PSMA, HER2, WT1, EphA3, EGFR, CD20, MAGE, BAGE, GAGE, NY-ESO-1, Telomerase, Survivin, or any combination thereof.

Various aspects of the disclosure are described in further detail below. Additional definitions are set out throughout the specification.

### Pluripotent Stem Cells

In various embodiments, hematopoietic cells can be produced from human pluripotent stem cells (hPSCs), including but not limited to human embryonic stem cells (hESCs), human parthenogenetic stem cells (hpSCs), nuclear transfer derived stem cells, and induced pluripotent stem cells (iPSCs). Methods of obtaining such hPSCs are well known in the art.

Pluripotent stem cells are defined functionally as stem cells that are: (a) capable of inducing teratomas when transplanted in immunodeficient (SCID) mice; (b) capable of differentiating to cell types of all three germ layers (e.g., ectodermal, mesodermal, and endodermal cell types); and (c) express one or more markers of embryonic stem cells (e.g., OCT4, alkaline phosphatase, SSEA-3 surface antigen, SSEA-4 surface antigen, NANOG, TRA-1-60, TRA-1-81, SOX2, REX1, etc.). In certain embodiments, pluripotent stem cells express one or more markers selected from the group consisting of OCT4, alkaline phosphatase, SSEA-3, SSEA-4, TRA-1-60, and TRA-1-81. Exemplary pluripotent stem cells can be generated using, for example, methods known in the art. Exemplary pluripotent stem cells include embryonic stem cells derived from the ICM of blastocyst stage embryos, as well as embryonic stem cells derived from one or more blastomeres of a cleavage stage or morula stage embryo (optionally without destroying the remainder of the embryo). Such embryonic stem cells can be generated from embryonic material produced by fertilization or by asexual means, including somatic cell nuclear transfer (SCNT), parthenogenesis, and androgenesis. Further exemplary pluripotent stem cells include induced pluripotent stem cells (iPSCs) generated by reprogramming a somatic cell by expressing a combination of factors (herein referred to as reprogramming factors). The iPSCs can be generated using fetal, postnatal, newborn, juvenile, or adult somatic cells.

In certain embodiments, factors that can be used to reprogram somatic cells to pluripotent stem cells include, for example, a combination of OCT4 (sometimes referred to as OCT3/4), SOX2, c-Myc, and Klf4. In other embodiments, factors that can be used to reprogram somatic cells to pluripotent stem cells include, for example, a combination of OCT4, SOX2, NANOG, and LIN28. In certain embodiments, at least two reprogramming factors are expressed in a somatic cell to successfully reprogram the somatic cell. In other embodiments, at least three reprogramming factors are expressed in a somatic cell to successfully reprogram the somatic cell. In other embodiments, at least four reprogramming factors are expressed in a somatic cell to successfully reprogram the somatic cell. In other embodiments, additional reprogramming factors are identified and used alone or in combination with one or more known reprogramming factors to reprogram a somatic cell to a pluripotent stem cell. Induced pluripotent stem cells are defined functionally and include cells that are reprogrammed using any of a variety of methods (integrative vectors, non-integrative vectors, chemical means, etc). Pluripotent stem cells may be genetically modified or otherwise modified to increase longevity, potency, homing, to prevent or reduce alloimmune responses, or to deliver a desired factor in cells that are differentiated from such pluripotent cells.

Induced pluripotent stem cells (iPS cells or iPSC) can be produced by protein transduction of reprogramming factors in a somatic cell. In certain embodiments, at least two reprogramming proteins are transduced into a somatic cell to successfully reprogram the somatic cell. In other embodiments, at least three reprogramming proteins are transduced into a somatic cell to successfully reprogram the somatic cell. In other embodiments, at least four reprogramming proteins are transduced into a somatic cell to successfully reprogram the somatic cell.

The pluripotent stem cells can be from any species. Embryonic stem cells have been successfully derived in, for example, mice, multiple species of non-human primates, and humans, and embryonic stem-like cells have been generated from numerous additional species. Thus, one of skill in the art can generate embryonic stem cells and embryo-derived stem cells from any species, including but not limited to, human, non-human primates, rodents (mice, rats), ungulates (cows, sheep, etc), dogs (domestic and wild dogs), cats (domestic and wild cats such as lions, tigers, cheetahs), rabbits, hamsters, gerbils, squirrel, guinea pig, goats, elephants, panda (including giant panda), pigs, raccoon, horse, zebra, marine mammals (dolphin, whales, etc.) and the like. In certain embodiments, the species is an endangered species. In certain embodiments, the species is a currently extinct species.

Similarly, iPS cells can be from any species. These iPS cells have been successfully generated using mouse and human cells. Furthermore, iPS cells have been successfully generated using embryonic, fetal, newborn, and adult tissue. Accordingly, one can readily generate iPS cells using a donor cell from any species. Thus, one can generate iPS cells from any species, including but not limited to, human, non-human primates, rodents (mice, rats), ungulates (cows, sheep, etc), dogs (domestic and wild dogs), cats (domestic and wild cats such as lions, tigers, cheetahs), rabbits, hamsters, goats, elephants, panda (including giant panda), pigs, raccoon, horse, zebra, marine mammals (dolphin, whales, etc.) and the like. In certain embodiments, the species is an endangered species. In certain embodiments, the species is a currently extinct species.

Induced pluripotent stem cells can be generated using, as a starting point, virtually any somatic cell of any developmental stage. For example, the cell can be from an embryo, fetus, neonate, juvenile, or adult donor. Exemplary somatic cells that can be used include fibroblasts, such as dermal fibroblasts obtained by a skin sample or biopsy, synoviocytes from synovial tissue, foreskin cells, cheek cells, or lung fibroblasts. Although skin and cheek provide a readily available and easily attainable source of appropriate cells, virtually any cell can be used. In certain embodiments, the somatic cell is not a fibroblast.

The induced pluripotent stem cell may be produced by expressing or inducing the expression of one or more reprogramming factors in a somatic cell. The somatic cell may be a fibroblast, such as a dermal fibroblast, synovial fibroblast, or lung fibroblast, or a non-fibroblastic somatic cell. The somatic cell may be reprogrammed through causing expression of (such as through viral transduction, integrating or non-integrating vectors, etc.) and/or contact with (e.g., using protein transduction domains, electroporation, microinjection, cationic amphiphiles, fusion with lipid bilayers containing, detergent permeabilization, etc.) at least 1, 2, 3, 4, 5 reprogramming factors. The reprogramming factors may be selected from OCT3/4, SOX2, NANOG, LIN28, C-MYC, and KLF4. Expression of the reprogramming factors may be induced by contacting the somatic cells with at least one agent, such as a small organic molecule agent, that induce expression of reprogramming factors.

Further exemplary pluripotent stem cells include induced pluripotent stem cells generated by reprogramming a somatic cell by expressing or inducing expression of a combination of factors (“reprogramming factors”). iPS cells may be obtained from a cell bank. The making of iPS cells may be an initial step in the production of differentiated cells. iPS cells may be specifically generated using material from a particular patient or matched donor with the goal of generating tissue-matched hematopoietic cells. iPSCs can be produced from cells that are not substantially immunogenic in an intended recipient, e.g., produced from autologous cells or from cells histocompatible to an intended recipient.

The somatic cell may also be reprogrammed using a combinatorial approach wherein the reprogramming factor is expressed (e.g., using a viral vector, plasmid, and the like) and the expression of the reprogramming factor is induced (e.g., using a small organic molecule.) For example, reprogramming factors may be expressed in the somatic cell by infection using a viral vector, such as a retroviral vector or a lentiviral vector. Also, reprogramming factors may be expressed in the somatic cell using a non-integrative vector, such as an episomal plasmid. See, e.g., Yu et al., Science. 2009 May 8; 324(5928):797-801, which is hereby incorporated by reference in its entirety. When reprogramming factors are expressed using non-integrative vectors, the factors may be expressed in the cells using electroporation, transfection, or transformation of the somatic cells with the vectors. For example, in mouse cells, expression of four factors (OCT3/4, SOX2, C-MYC, and KLF4) using integrative viral vectors is sufficient to reprogram a somatic cell. In human cells, expression of four factors (OCT3/4, SOX2, NANOG, and LIN28) using integrative viral vectors is sufficient to reprogram a somatic cell.

Once the reprogramming factors are expressed in the cells, the cells may be cultured. Over time, cells with ES characteristics appear in the culture dish. The cells may be chosen and subcultured based on, for example, ES morphology, or based on expression of a selectable or detectable marker. The cells may be cultured to produce a culture of cells that resemble ES cells—these are putative iPS cells.

To confirm the pluripotency of the iPS cells, the cells may be tested in one or more assays of pluripotency. For example, the cells may be tested for expression of ES cell markers; the cells may be evaluated for ability to produce teratomas when transplanted into SCID mice; the cells may be evaluated for ability to differentiate to produce cell types of all three germ layers. Once a pluripotent iPSC is obtained it may be used to produce cell types disclosed herein.

Another method of obtaining hPSCs is by parthenogenesis. “Parthenogenesis” (“parthenogenically activated” and “parthenogenetically activated” are used herein interchangeably) refers to the process by which activation of the oocyte occurs in the absence of sperm penetration, and refers to the development of an early stage embryo comprising trophectoderm and inner cell mass that is obtained by activation of an oocyte or embryonic cell, e.g., blastomere, comprising DNA of all female origin. In a related aspect, a “parthenote” refers to the resulting cell obtained by such activation. In another related aspect, “blastocyst: refers to a cleavage stage of a fertilized of activated oocyte comprising a hollow ball of cells made of outer trophoblast cells and an inner cell mass (ICM). In a further related aspect, “blastocyst formation” refers to the process, after oocyte fertilization or activation, where the oocyte is subsequently cultured in media for a time to enable it to develop into a hollow ball of cells made of outer trophoblast cells and ICM (e.g., 5 to 6 days).

Another method of obtaining hPSCs is through nuclear transfer. As used herein, “nuclear transfer” refers to the fusion or transplantation of a donor cell or DNA from a donor cell into a suitable recipient cell, typically an oocyte of the same or different species that is treated before, concomitant, or after transplant or fusion to remove or inactivate its endogenous nuclear DNA. The donor cell used for nuclear transfer include embryonic and differentiated cells, e.g., somatic and germ cells. The donor cell may be in a proliferative cell cycle (G1, G2, S or M) or non-proliferating (GO or quiescent). Preferably, the donor cell or DNA from the donor cell is derived from a proliferating mammalian cell culture, e.g., a fibroblast cell culture. The donor cell optionally may be transgenic, i.e., it may comprise one or more genetic addition, substitution, or deletion modifications.

A further method for obtaining hPSCs is through the reprogramming of cells to obtain induced pluripotent stem cells. Takahashi et al. (Cell 131, 861-872 (2007)) have disclosed methods for reprogramming differentiated cells, without the use of any embryo or ES (embryonic stem) cell, and establishing an inducible pluripotent stem cell having similar pluripotency and growing abilities to those of an ES cell. Nuclear reprogramming factors for differentiated fibroblasts include products of the following four genes: an Oct family gene; a Sox family gene; a Klf family gene; and a Myc family gene.

The pluripotent state of the cells is preferably maintained by culturing cells under appropriate conditions, for example, by culturing on a fibroblast feeder layer or another feeder layer or culture that includes leukemia inhibitory factor (LIF). The pluripotent state of such cultured cells can be confirmed by various methods, e.g., (i) confirming the expression of markers characteristic of pluripotent cells; (ii) production of chimeric animals that contain cells that express the genotype of the pluripotent cells; (iii) injection of cells into animals, e.g., SCID mice, with the production of different differentiated cell types in vivo; and (iv) observation of the differentiation of the cells (e.g., when cultured in the absence of feeder layer or LIF) into embryoid bodies and other differentiated cell types in vitro.

The pluripotent state of the cells used in the present disclosure can be confirmed by various methods. For example, the cells can be tested for the presence or absence of characteristic ES cell markers. In the case of human ES cells, examples of such markers are identified supra, including SSEA-4, SSEA-3, TRA-1-60, TRA-1-81 and OCT 4, and are known in the art.

Also, pluripotency can be confirmed by injecting the cells into a suitable animal, e.g., a SCID mouse, and observing the production of differentiated cells and tissues. Still another method of confirming pluripotency is using the subject pluripotent cells to generate chimeric animals and observing the contribution of the introduced cells to different cell types.

Yet another method of confirming pluripotency is to observe ES cell differentiation into embryoid bodies and other differentiated cell types when cultured under conditions that favor differentiation (e.g., removal of fibroblast feeder layers). This method has been utilized and it has been confirmed that the subject pluripotent cells give rise to embryoid bodies and different differentiated cell types in tissue culture.

hPSCs can be maintained in culture in a pluripotent state by routine passage until it is desired that hematopoietic lineage cells be derived.

### 3D Matrix- and Carrier-free Sphere Culture to Produce Hematopoietic Cells

Hematopoietic stem cells (HSC) give rise to cells of all hematopoietic lineages. Significant progress has been made on how to make hematopoietic cells from PSCs. However, processes suitable for large scale industrial manufacture are still unavailable, a clear obstacle for translating stem cells into clinical application.

Provided herein, in some embodiments, is a highly reproducible, scalable and defined 3D sphere differentiation system to convert human PSCs into HECs as well as HPCs, which, in turn, can be robustly differentiated into almost all lineages of hematopoietic cells including, but not limited, to MKs/platelets, RBCs, and NK cells.

Compare to previously reported methods, the 3D sphere system disclosed herein has significant advantages in the following technical aspects, without limitation:

(1) Well-controlled PSC sphere sizes at initiation of differentiation, which is critical for homogenous specification of human PSCs toward mesoderm lineage with high efficiency and small variability. The uniformity with desirable sphere sizes can allow oxygen, nutrients and differentiation inducing factors/molecules to penetrate the central core of spheres and result in a synchronized differentiation process for generating pure lineage specific populations, which the spontaneously formed embryoid bodies (EB) and other so-called organoid systems lack. The system of the present disclosure is suitable for HEC, HPC and hematopoietic cell production from different hESC or iPSC lines with minimum effort of sphere size optimization;

(2) No feeder cells, serum, undefined matrix or carrier is needed in the 3D sphere platform of the present disclosure, thus rendering it friendly to cGMP compliant cell manufacture for potential clinical application;

(3) The entire process of PSC expansion and differentiation is under 3D suspension culture condition, which can be readily scaled-up into commercially available single-use bioreactors at any desirable working volume;

(4) HPCs can be naturally and automatically released into suspension as single cells without any treatment such as enzymatic dissociation. The released HPCs maintained high viability which renders them with high tolerance for downstream processes such as volume reduction, filtration, cryopreservation, and enrichment/depletion if necessary;

(5) Other mesoderm lineage by-products such as mesenchymal stem cells (MSCs), endothelial cells and smooth muscle cells can be obtained from the 3D sphere platform of the present disclosure.

Various 3D sphere culture procedures can be used, such as include forced-floating methods that modify cell culture surfaces and thereby promote 3D culture formation by preventing cells from attaching to their surface; the hanging drop method which supports cellular growth in suspension; and agitation/rotary systems that encourage cells to adhere to each other to form 3D spheroids.

One method for generating 3D spheroids is to prevent their attachment to the vessel surface by modifying the surface, resulting in forced-floating of cells. This promotes cell-cell contacts which, in turn, promotes multi-cellular sphere formation. Exemplary surface modification includes poly-2-hydroxyethyl methacrylate (poly-HEMA) and agarose.

The hanging drop method of 3D spheroid production uses a small aliquot (typically 20 ml) of a single cell suspension which is pipetted into the wells of a tray. Similarly to forced-floating, the cell density of the seeding suspension (e.g. 50, 100, 500 cells/well, among others) can be altered as relevant, depending on the required size of spheroids. Following cell seeding, the tray is subsequently inverted and aliquots of cell suspension turn into hanging drops that are kept in place due to surface tension. Cells accumulate at the tip of the drop, at the liquid-air interface, and are allowed to proliferate.

Agitation-based approaches for the production of 3D spheroids can be loosely placed into two categories as (i) spinner flask bioreactors and (ii) rotational culture systems. The general principle behind these methods is that a cell suspension is placed into a container and the suspension is kept in motion, that is, either it is gently stirred or the container is rotated. The continuous motion of the suspended cells means that cells do not adhere to the container walls, but instead form cell-cell interactions. Spinner flask bioreactors (typically known as “spinners”) include a container to hold the cell suspension and a stirring element to ensure that the cell suspension is continuously mixed. Rotating cell culture bioreactors function by similar means as the spinner flask bioreactor but, instead of using a stirring bar/rod to keep cell suspensions moving, the culture container itself is rotated.

In some embodiments, provided herein is a spinner flask based 3D sphere culture protocol. A plurality of hPSCs can be continuously cultured as substantially uniform spheres in spinner flasks with a defined culture medium in the absence of feeder cells and matrix. The culture medium can be any defined, xeno-free, serum-free cell culture medium designed to support the growth and expansion of hPSCs such as hiPSC and hES. In one example, the medium is NutriStem® medium (Biological Industry). In some embodiments, the medium can be mTeSR™1, mTeSR™2, TeSR™-E8™ medium (StemCell Technologies), or other stem cell medium. The medium can be supplemented with small molecule inhibitor of Rho-associated, coiled-coil containing protein kinase (ROCK) such as Y27632 or other ROCK inhibitors such as Thiazovivin, ROCK II inhibitor (e.g., SR3677) and GSK429286A. With this suspension culture system, hPSC cultures can be serially passaged and consistently expanded for at least 10 passages. A typical passaging interval for 3D-hiPSC sphere can be about 3-6 days, at which time spheres can grow into a size of about 230-260 μm in diameter. Sphere size can be monitored by taking an aliquot of the culture and observing using, e.g., microscopy. Then the spheres can be dissociated into single (or substantially single) cells using, e.g., an enzyme with proteolytic and collagenolytic activity for the detachment of primary and stem cell lines and tissues. In one example, the enzyme is Accutase® (Innovative Cell Technologies, Inc), or TrypLE (Thermo Fisher), or Trypsin/EDTA. Thereafter, the disassociated cells can be reaggregated to reform spheres in spinner flasks under continuous agitation at, e.g., 60-70RPM. Spheres gradually increased in size while maintaining a uniform structure together with a high pluripotency marker expression (OCT4) and a normal karyotype after at least 3-5 repeated passages. As used herein, a “passage” is understood to mean a cell sphere culture grown from single cells into spheres of a desirable size, at which time the spheres are disassociated into single cells and seeded again for the next passage. A passage can take about 3-6 days for 3D-hiPSC spheres, or longer or shorter, depending on the type of hPSCs and culturing conditions. Once sufficient amounts of 3D-hPSC spheres are obtained, they can be subject to 3D sphere differentiation, as described in more detail below.

In some embodiments, hiPSC cells can be cultured on a matrix such as Laminin 521 or Laminin 511 in NutriStem® hPSC XF medium (Biological Industries USA). Confluent and undifferentiated hiPSCs can be passaged using Accutase® or TripLE and seeded onto a surface coated with reduced (½) concentration of matrix at density of 6-8×10⁴ cells per cm² in NutriStem® supplemented with 1 μM of Y27632 and culture for 3-7 days. HiPSCs can be expanded in this condition for 3-5 passages, or for as many passages as needed. The undifferentiated status of hiPSCs can be quantitated with the expression level of Oct-4 by flow cytometry analysis (over 95% Oct-4 positive).

To initiate 3D suspension culture, confluent undifferentiated hiPSCs can be dissociated by Accutase or TripLE and were seeded into a spinner flask at a density of, e.g., 1×10⁶ cell/mL in NutriStem® supplemented with Y27632 (about 1 μM). The cells can be cultured uninterrupted for 48 hours with agitation rate of 50-80 RPM in a 30-mL spinner flask (Abel Biott). Forty-eight hours after seeding, a small sample can be taken out, and the morphology and sphere sizes can be examined by microscopy. Periodically media can be refreshed until sphere sizes reached 250-300 micrometers in diameter. For passaging, hiPSC spheres can be washed with PBS (Mg⁻, Ca⁻), and then dissociated by an enzyme such as Accutase or TripLE. Dissociated hiPSC single cells can then be seeded at a desired density for either expansion or initiation of hematopoietic differentiation.

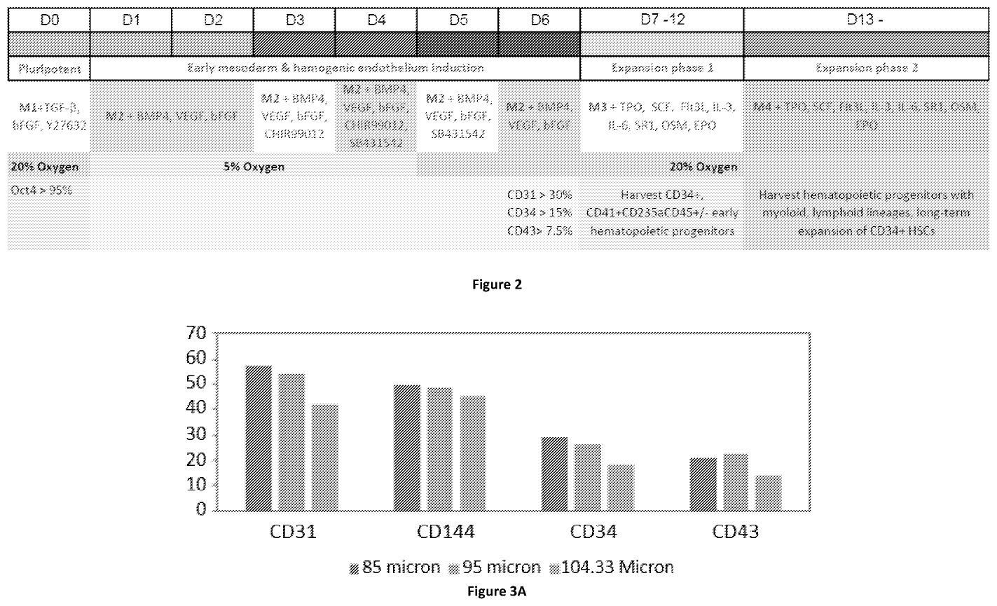

To generate HECs and hematopoietic lineages from hPSCs, 3D-hPSC spheres in suspension can be directly induced in a stepwise fashion with defined growth factors and small molecules (FIG. 2). In some embodiments, this can be done in 3D spinner flasks, or other 3D sphere culturing methods. In various embodiments, continuous 3D sphere culture can be integrated with several dissociation/reaggregation steps, while growth factors and small molecules can be added at different stages to induce differentiation.

As shown in FIG. 2, hPSCs (e.g., hiPSCs) can be seeded as single cells at a desired density (e.g., 0.5-1.5×10⁶ cells/ml, depending on cell size) in HEC induction medium M1 (e.g., NutriStem®, mTeSk™1, mTeSk™2, TeSR™-E8™ or other culture medium suitable for 3D suspension culture) supplemented with Y27632 (about 1 μM), for about 6-24 or about 12 hours till desirable sphere size. Typical sphere sizes can be between 60-150 micrometers, about 70-120 micrometers or about 80-100 micrometers in diameter depending on seeding densities. Without wishing to be bound by theory, it is believed that the sphere size can affect HEC differentiation due to geometry, cell-to-cell contact, as well as accessibility to nutrients and growth factors that can form a gradient outside the spheres. In some embodiments, sphere size can be monitored e.g., using microscopy, to be in the range of about 60-110 micrometers, about 70-100 micrometers or about 80-90 micrometers in diameter before initiating HEC differentiation.

To initiate HEC differentiation, M1 can be removed and replaced with the HEC induction medium M2 (e.g., growth factor-free NutriStem® hPSC XF Medium®, mTeSR™1, mTeSR™2, TeSR™-E8™ or other culture medium suitable for promoting mesoderm differentiation in 3D suspension culture) supplemented with BMP4, VEGF, and bFGF at a concentration of about 10-100, about 25-50, or about 30-40 ng/mL. HiPSC spheres in M2 can be cultured under hypoxia condition (about 5% oxygen) for about 1-10 days or 3-8 days or 4 days, followed by about 1-5 or about 2 additional days at normal oxygen concentration of about 20%. Without wishing to be bound by theory, it is believed that the hypoxia condition can mimicking early embryonic development condition, thereby inducing differentiation.

Small molecule CHIR99021 can be added at about 1-10, about 2-5, or about 3 μM after the cells have spent some time (e.g., 1-5 days) under hypoxia condition Small molecule SB431542 can be added, together with or following CHIR99021 (e.g., 0-3 days after CHIR99021 addition), at about 1-10, about 2-5, or about 3 μM. In the example shown in FIG. 2, CHIR99021 is added for Day 3 and 4, and SB431542 Day 4 and 5. Thereafter, CHIR99021 and SB431542 can be removed from the culture medium.

During late stages (e.g., on Day 6 or later) of HEC differentiation, cell spheres can be dissociated into substantially single cell suspension by treatment of enzyme (e.g., Accutase®, TrypLE, or Trypsin/EDTA for 15-30 minutes at 37° C.). The expression of HEC specific surface markers CD31, CD144 (VE-Cadherin), CD34, and CD43 can be analyzed using flow cytometry. The substantially single cells of HECs can be seeded into a scaffold that mimics in vivo hematopoietic niche. The niche can be mimicked by culturing the cells in the presence of biomaterials, such as matrices, scaffolds, and culture substrates that represent key regulatory signals controlling cell fate. The biomaterials can be natural, semi-synthetic and synthetic biomaterials, and/or mixtures thereof. Suitable synthetic materials for the scaffold include polymers selected from porous solids, nanofibers, and hydrogels, such as chitosan, polylactic acid, polystyrene, peptides including self-assembling peptides, hydrogels composed of polyethylene glycol phosphate, polyethylene glycol fumarate, polyacrylamide, polyhydroxyethyl methacrylate, polycellulose acetate, and/or co-polymers thereof (see, for example, Saha et al., 2007, Curr. Opin. Chem. Biol. 11(4): 381-387; Saha et al., 2008, Biophysical Journal 95: 4426-4438; Little et al.; 2008, Chem. Rev. 108, 1787-1796; Carletti et al., Methods Mol Biol. 2011; 695: 17-39; Geckil et al., Nanomedicine (Lond). 2010 April; 5(3): 469-484; all incorporated herein by reference in its entirety). Once seeded, the cells can be cultured, within the scaffold and in the presence of a suitable medium and suitable growth factors, to differentiate into desirable lymphoid lineage cells such as lymphocytes (such as T lymphocytes), natural killer (NK) cells, common myeloid progenitor cells, common granulomonocytic progenitor cells, monocytes, macrophages, and/or dendritic cells. One of ordinary skill in the art would appreciate the selection of suitable medium and suitable growth factors in accordance with desirable lymphoid lineage cells.

Alternatively, the HEC-containing spheres (without enzymatic disassociation) can be transitioned into hematopoietic commitment and expansion medium M3 (basal media such as StemSpan™-ACF (STEMCELL Technologies Inc.), PRIME-XV® (Irving Scientific), PromoCell® Hematopoietic Progenitor Expansion medium DXF (PromoCell GmbH) and other culture system suitable for hematopoietic stem cell expansion in 3D suspension culture) to induce differentiation into and expansion of hematopoietic progenitor cells (HPCs). M3 can be supplemented with one or more of TPO (10-25 ng/ml), SCF (10-25 ng/ml), Flt3L (10-25 ng/ml), IL-3 (2-10 ng/ml), IL-6 (2-10 ng/ml), SR1 (0.75 μM), OSM (2-10 ng/ml), and EPO (2 U/ml) for about 3-10 days, about 4-8 days or about 5 days of phase 1 expansion. HPCs can be automatically (without enzymatic disassociation of spheres) released from the spheres.

Further differentiation and expansion can be achieved in the hematopoietic differentiation/expansion medium M4 (basal media such as StemSpan™-ACF (STEMCELL Technologies Inc.), PRIME-XV® (Irving Scientific), PromoCell® Hematopoietic Progenitor Expansion medium DXF (PromoCell GmbH) and other culture system suitable for lineage-specific expansion and maturation of variety of hematopoietic cells of megakaryocytic, erythroid, myeloid and lymphoid lineages in 3D suspension culture). M4 can be supplemented with one or more of TPO (10-25 ng/ml), SCF (10-25 ng/ml), Flt3L (10-25 ng/ml), IL-3 (2-10 ng/ml), IL-6 (2-10 ng/ml), SR1 (0.75 μM), OSM (2-10 ng/ml), and EPO (3 U/ml) for such phase 2 expansion (up to 40 days or longer). One of ordinary skill in the art would understand that different media and growth factors can be used to promote differentiation into different cell types, such as common erythroid/megakaryocytic progenitor cells, erythrocytes, megakaryocytes, platelets, common lymphoid progenitor cells, lymphoid lineage cells, lymphocytes (such as T lymphocytes), natural killer (NK) cells, common myeloid progenitor cells, common granulomonocytic progenitor cells, monocytes, macrophages, and/or dendritic cells, or a mixture of any two or more of the foregoing.

Media can be changed daily during differentiation. When switching from a first medium to a second medium, gradual adaptation to the second medium can be achieved through a dilution series of the first medium and the second medium. For example, gradual adaption from 100% the first medium to 100% the second medium can include intermediate culturing with the first medium and the second medium sequentially at 75%:25%, 50%:50%, and 25%:75%, with the cells spending 2-6 days in each medium composition. Other dilution series can also be used.

In various embodiments, provided herein is a new, efficient and defined 3D sphere platform to generate desirable cells from hPSCs, specifically HECs and hematopoietic cells that can be used for cell therapy for various purposes.

### Use of Hematopoietic Cells

Importantly, as demonstrated herein, the HPCs generated with the 3D PSC differentiation system of the present disclosure possess the capacity to form multiple cell types of all blood lineages, especially the CD34⁺ population, which can robustly give rise to multiple types of CFUs, resembling the characteristics of multipotential HSCs. Furthermore, after culturing under specific conditions, the CD235a⁺CD41⁺ double positive HPCs, which may represent a common progenitor for MK and erythroid cells, preferentially generated MKs/platelets and erythroid cells, respectively. Human PSC-derived MKs/platelets and RBCs can be used not only for transfusion therapy but can also serve as carriers for therapeutic proteins. To achieve this goal, master PSC banks can be engineered to express therapeutic proteins for manufacture of MKs/platelets which can release therapeutic proteins upon activation at the site of wound or tumor, etc. As the platelet α-granule signal sequence has been characterized, genes encoding therapeutic recombinant fusion proteins can be introduced into PSC. After differentiating into MK cells these proteins will be packaged into α-granules and released at desirable sites to achieve therapeutic purposes. These proteins include, but not limited to, factor VIII for treatment of hemophilia by localized delivery at site of injury; erythropoietin for acceleration of fibrin-induced wound-healing response, such as in the treatment of diabetic ulcers and burns; and insulin-like growth factor 1, basic fibroblast growth factor, anti-angiogenic/anti-tumor proteins; etc. Similarly, engineered master PSC banks for manufacturing universal RhD negative 0 type RBCs can be used to generate universal RBCs expressing therapeutic proteins, e.g., proteins involved in the induction of antigen-specific immune tolerance. Universal RBCs expressing specific antigens on their surfaces or inside the cells can be transplanted into super-sensitive individuals. As RBCs circulate, age and are cleared, the specific antigens will be processed using the immune system's natural mechanisms to prevent autoimmunity.

The acquisition of lymphoid lineage potential has long been regarded as an important indicator of definitive hematopoiesis within the aorta-gonad-mesonephros (AGM) region in contrast to primitive hematopoiesis in yolk sac within the embryo (Park et al. 2018). As shown in the Examples herein, HPCs obtained from the 3D differentiation PSC spheres of the present disclosure generated CD56^{+high} NK cells, which suggests the defined system of the present disclosure supports the development of definitive hematopoiesis. Several previous reports have shown the generation of lymphoid cells, but most of these studies used feeder cells and/or serum (de Pooter and Zuniga-Pflucker 2007; D'Souza et al. 2016; Zeng et al. 2017; Ditadi et al. 2015), which limits the potential clinical application.

Therefore, another significant technological advance of the present disclosure is the generation of pure bona fide NK cells in a serum- and feeder-free 3D condition. This makes it feasible to manufacture clinically relevant dose of NK cells from PSCs (e.g., hESCs and iPSCs) which may carry Chimeric Antigen Receptors (CAR) targeting tumor specific antigens for cancer immunotherapy. Adoptive cell therapy utilizing engineered CAR-T cells have shown to be clinically successful in treating patients with B-cell malignancy (Grupp et al. 2013; Kochenderfer et al. 2010). CAR-T cells, however, have severe limitation due to the autologous T cell manufacturing process and transfusion as risk of serious graft-versus-host disease (GVHD) may be incurred with the infusion of allogenic T cells (Mehta and Rezvani 2018). Unlike T cells and B cells, NK cells do not express rearranged, antigen-specific receptors. NK cell receptors are germline encoded, with either activating or inhibitory function upon binding with their specific ligands on target cells. KIRs are the most studied NK cell receptors that recognize HLA class I molecules. Other receptors such as NKG2A, -B, -C, -D, -E and -F recognize non-classical HLA class I molecules (HLA-E). Healthy cells are protected from NK cells by the recognition of “self” HLA molecules on their surface through inhibitory NK receptors (Lanier 2001; Yokoyama 1998). Tumor or virus infected cells often downregulate or lose their HLA molecules as camouflage to evade attack by T cells (Costello, Gastaut, and Olive 1999; Algarra et al. 2004). Early clinical investigations of autologous NK cell adoptive therapy proved to be ineffective in cancer treatment (Burns et al. 2003; deMagalhaes-Silverman et al. 2000). However, the clinical benefits of alloreactive NK cells in HSC transplantation (Ruggeri et al. 2002) and cancer therapy (Bachanova et al. 2014) demonstrated promising results. Therefore CAR-NK cells are believed to be a superior choice than CAR-T for allogeneic cell therapy.

The advancement of CAR-NK, however, has been hampered by the limited NK cell sources. NK cells can be collected from peripheral blood (PB), bone marrow (BM), and umbilical cord blood (CB). The process is cumbersome and may cause unwanted health risks to donors (Winters 2006; Yuan et al. 2010). Harvested NK cells have limited expansion capability and contamination by small amounts of T cells or B cells may cause GVHD. NK cells harvested from CB has been used in ongoing clinical trials, but they must be expanded significantly by co-culture with GMP-grade artificial antigen presenting cells (Shah et al. 2013). Cell line NK-92 is used in several CAR-NK clinical trials (in China). NK-92 cell line was derived from a patient with NK cell lymphoma. These cells can be EBV positive and carry multiple cytogenetic abnormality found in lymphoma (MacLeod et al. 2002). NK-92 derived CAR-NK cells, therefore, must be irradiated before infusion to patients, which has negative impact on their in vivo persistence and function (Schonfeld et al. 2015) Human PSCs (both hESCs and iPSCs) have been proven to be capable of generating NK cells (Knorr et al. 2013; Li et al. 2018; Zeng et al. 2017). Early reported studies depended on spin EB generation ((Knorr et al. 2013; Li et al. 2018), which is unsuitable for scaled-up processes. Xeno-origin feeder-cells were used for PSC culture (Knorr et al. 2013) and NK differentiation (Zeng et al. 2017). Our newly developed 3D NK manufacture process, which combines 3D sphere differentiation with 3D scaffolds mimicking the microenvironments of organ architecture, has significant advantages over previous reported processes: (1) no limitation in scalability; (2) our NK-specific culture medium is defined, serum-free, and feeder-free; (3) the NK population is pure with no contamination of T-cell and B-cells. We have also established hiPSC lines that do not express HLA class I molecules (A, B, C) but express non-classic class I molecule HLA-E. Through engineering NK-tailored CARs into such hiPSC lines to establish master PSC banks, we can generate universal CAR-NK cells for truly off-the-shelf therapeutic products.

Thus, provided herein, in addition to a robust and defined 3D sphere platform to generate HECs and HPCs from renewable hPSCs, are lineage specific hematopoietic cells derived therefrom. This system is not only amenable to large-scale production efforts, but also eliminated dependence on feeder cells, animal serum, and matrix, thus rendering it friendly to cGMP compliant cell manufacturing protocol and making the process more amenable to clinical translation. Using either single or integrated multi-stage bioreactors, any hematopoietic cells can be manufactured on-demand. The applications for such technical advances will be limitless, as one of ordinary skill in the art would appreciate.

In some embodiments, the cell compositions provided herein can be used in cell therapy. The cell therapy can be selected from, e.g., an adoptive cell therapy, CAR-T cell therapy, engineered TCR T cell therapy, a tumor infiltrating lymphocyte therapy, an antigen-trained T cell therapy, or an enriched antigen-specific T cell therapy.

In some embodiments, the cell composition can be formulated in pharmaceutically-acceptable amounts and in pharmaceutically-acceptable compositions. The term “pharmaceutically acceptable” means a non-toxic material that does not interfere with the effectiveness of the biological activity of the active ingredients (e.g., biologically-active proteins of the nanoparticles). Such compositions may, in some embodiments, contain salts, buffering agents, preservatives, and optionally other therapeutic agents. Pharmaceutical compositions also may contain, in some embodiments, suitable preservatives. Pharmaceutical compositions may, in some embodiments, be presented in unit dosage form and may be prepared by any of the methods well-known in the art of pharmacy. Pharmaceutical compositions suitable for parenteral administration, in some embodiments, comprise a sterile aqueous or non-aqueous preparation of the nanoparticles, which is, in some embodiments, isotonic with the blood of the recipient subject. This preparation may be formulated according to known methods. A sterile injectable preparation also may be a sterile injectable solution or suspension in a non-toxic parenterally-acceptable diluent or solvent.

The compositions disclosed herein have numerous therapeutic utilities, including, e.g., the treatment of cancers, autoimmune diseases and infectious diseases. Methods described herein include treating a cancer in a subject by using the cells as described herein. Also provided are methods for reducing or ameliorating a symptom of a cancer in a subject, as well as methods for inhibiting the growth of a cancer and/or killing one or more cancer cells. In embodiments, the methods described herein decrease the size of a tumor and/or decrease the number of cancer cells in a subject administered with a described herein or a pharmaceutical composition described herein.

In embodiments, the cancer is a hematological cancer. In embodiments, the hematological cancer is leukemia or lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In embodiments, the cells are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition to be administered can be determined by a physician with consideration of individual differences in tumor size, extent of infection or metastasis, age, weight, and condition of the subject. In embodiments, the pharmaceutical composition described herein can be administered at a dosage of 10⁴ to 10⁹ cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the cells are administered to the subject parenterally. In embodiments, the cells are administered to the subject intravenously, subcutaneously, intratumorally, intranodally, intramuscularly, intradermally, or intraperitoneally. In embodiments, the cells are administered, e.g., injected, directly into a tumor or lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation.

In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### EXAMPLES

### Example 1: 3D Sphere Differentiation Suitable for all Hematopoietic Lineages

Transition of hiPSCs from 2D to 3D Suspension Culture

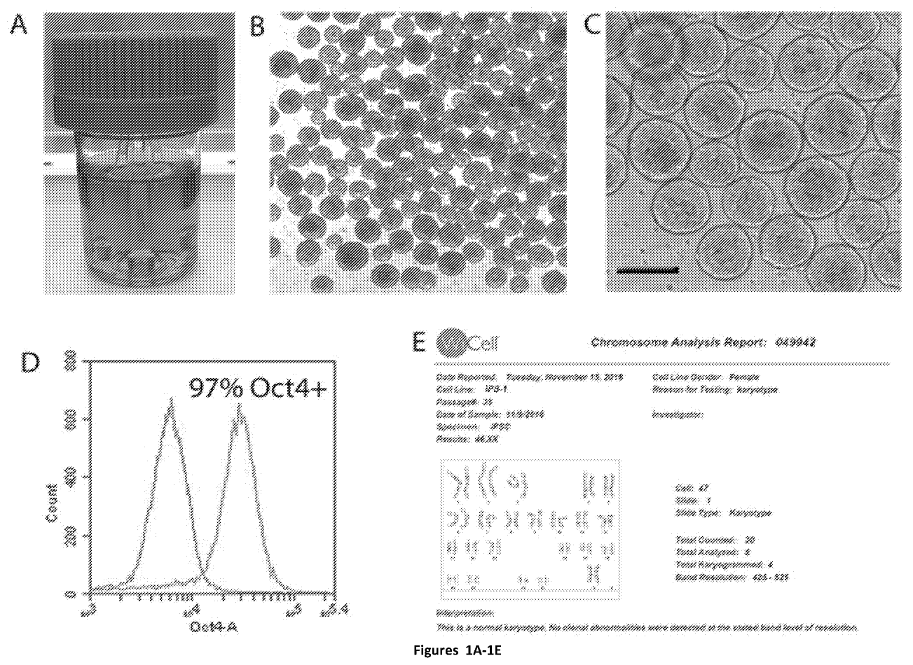

FIG. 1A illustrates a typical small bioreactor that was used in the present disclosure. Spinner flasks with working volumes between 250 ml to 3 L can also be used for larger scale experiments. A successful transition of 2D hiPSC cultures into 3D suspension cultures was characterized by the formation and subsequent growth of hiPSCs in the form of round-shape spheres as shown in FIGS. 1B and 1C. To monitor the pluripotency of the 3D transitioned hiPSCs, expression of the pluripotency marker Oct-4 was measured by flow cytometry. High quality undifferentiated pluripotent stem cells are Oct-4 positive (>95%, FIG. 1D). HiPSCs cultured under 3D spheres also have normal karyotype (FIG. 1E).

Stepwise Induction of hiPSCs into Hemogenic Endothelial and Hematopoietic Lineages

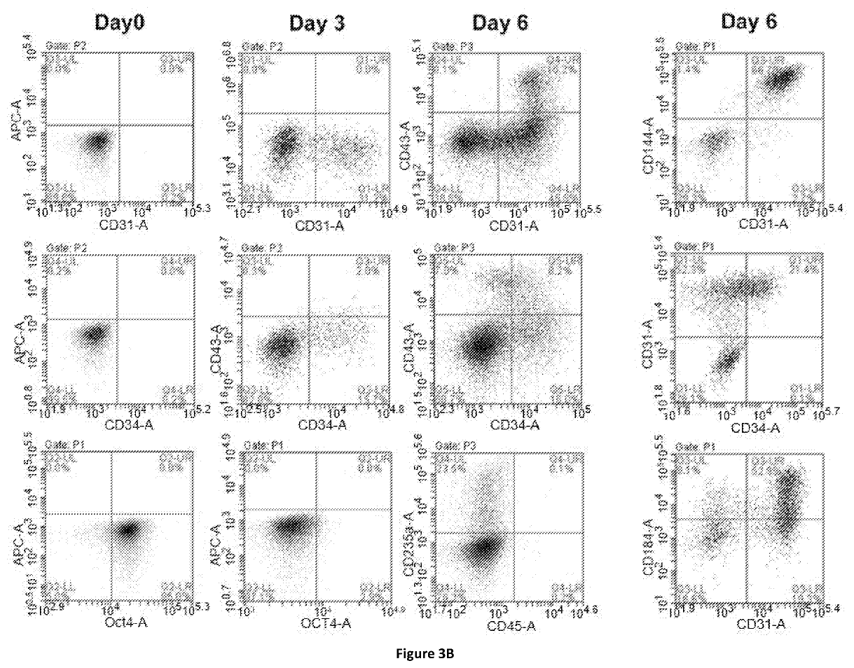

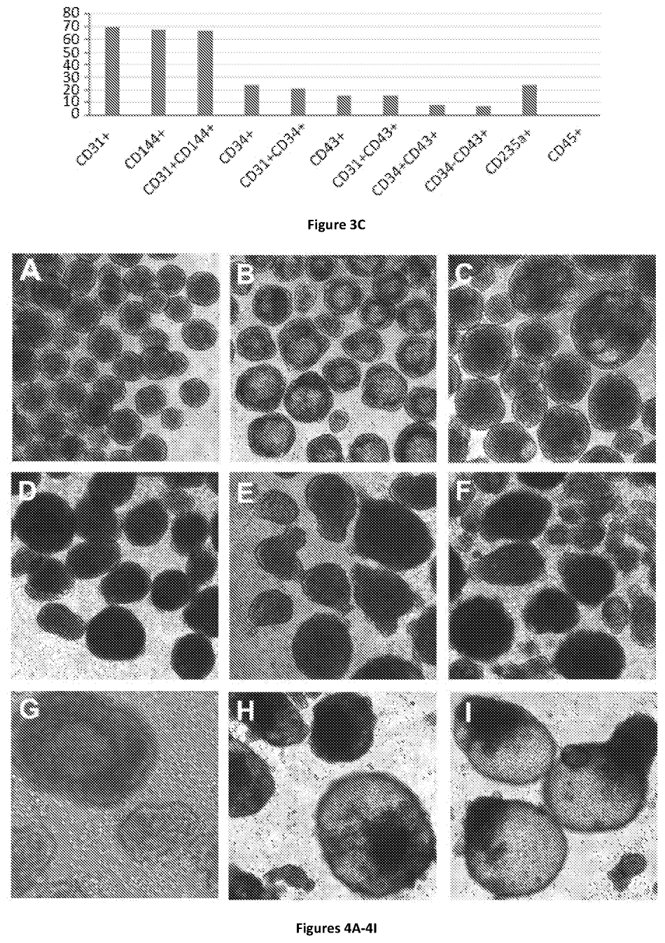

The strategy to induce hiPSCs toward HECs and HPCs is illustrated in FIG. 2. To obtain high yield and a pure HEC population, it is very important to only use 3D transitioned hiPSCs that are >95% positive for Oct-4 expression (as shown in FIGS. 1D and 3B). For each individual cell line, it is important to first determine the optimal sphere size at the start of the HEC induction. As shown in FIG. 3A, representative results from one hiPSC line demonstrated that starting from sphere sizes of 80-85 micrometers (in diameter) achieved higher HEC generation efficiency than spheres with sizes over 100 micrometers. Therefore, most differentiation indicated in this study started with sphere sizes between 80-85 micrometers.

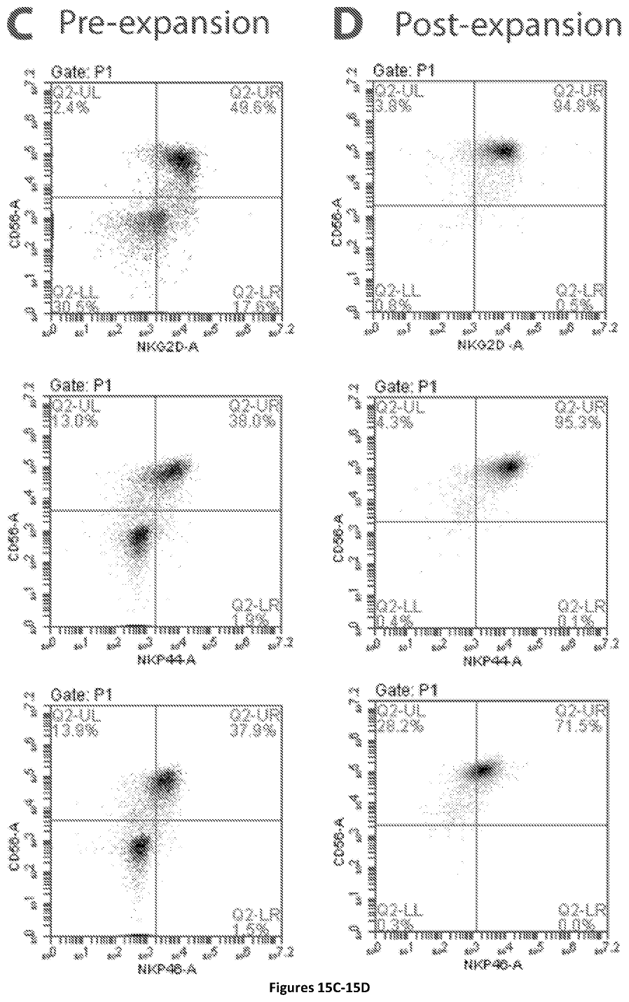

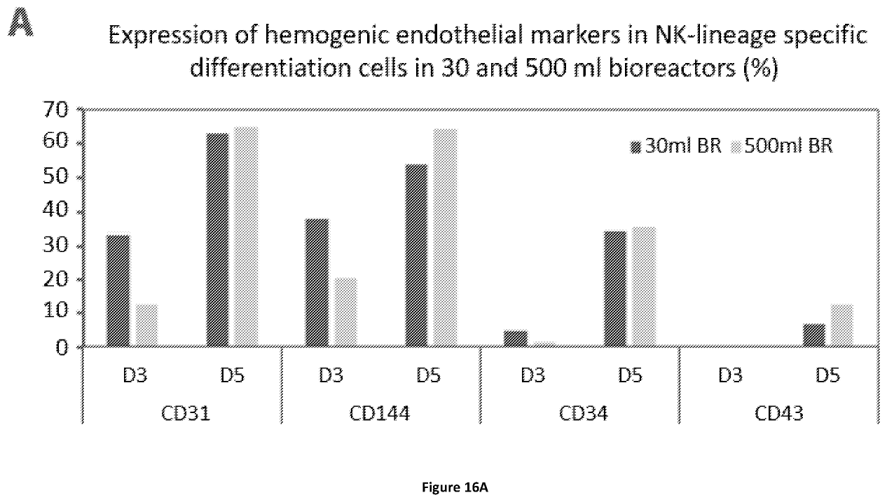

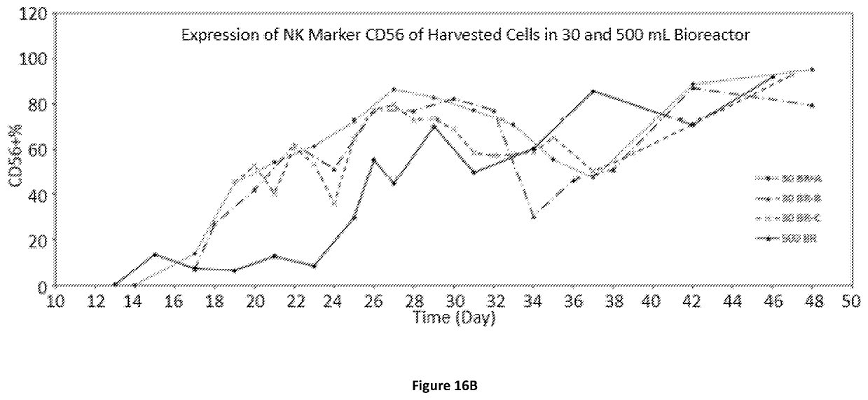

The efficiency of HEC generation was mainly monitored by expression of typical HEC markers CD31, CD144, CD34, and CD184 as well as hematopoietic marker CD43 to ensure that the HEC population will differentiate towards hematopoietic lineages. As shown in FIG. 3B, sphere cells prior to differentiation induction (day 0) showed no expression of CD31 and CD34 whereas 95% of them were Oct-4 positive. As early as day 3, a small but distinctive CD31⁺ population (31.2%) emerged, followed by CD34 expression (15.7%). CD43 expression (2%) was very low at this point. Oct-4 expression at this stage was already significantly reduced to 2.9%, confirming the loss of pluripotency. The HEC population normally reached its peak level at day 6 of the differentiating spheres. As shown in FIG. 3B, 66% of the whole population in suspension spheres were both CD31 and CD144 (VE-Cadherin) positive, both are markers for HECs. In addition, as shown in FIGS. 3B and 3C, 15.2% of CD31⁺ population were CD43⁺, indicating strong early commitment of HEC population to hematopoietic lineages. A significant CD34⁺ population (21.4%) also emerged from the CD31⁺ population. A fraction of HECs also expressed CD235a (23.5%) but almost no CD45 expression was detected, indicating early commitment of hematopoietic progenitors to erythroid lineages (Palis 2016). A majority of CD31⁺ HECs was also CD184⁺; however, some CD31⁻ cells were CD184⁺ as well. Interestingly, among the CD43^{+ population, commitment of hematopoietic lineages appeared to accompany a decrease of CD}34 expression. These results clearly confirm that our differentiation process is highly efficient in generating high quality HECs that are ideal for subsequent hematopoietic differentiation.

### Morphological Change of 3D Lineage-Specific Hematopoietic Differentiation

One of the major technical advantages of 3D suspension culture process is the capability of sampling and monitoring morphological changes at different stages of the long process. Significant morphological changes were observed throughout the whole differentiation process under 3D suspension condition. Undifferentiated hiPSC spheres were homogeneously round shaped with a small range of size variation (FIG. 4A). As early as day 3 of differentiation, size variation significantly increased with formation of cavity space inside most spheres (FIG. 4B). Spheres on day 6 (FIG. 4C) grew bigger (both size and internal cavity). From day 6 to day 9, a large quantity of suspension cells was present in culture medium, indicating the initiation of HPC release from spheres (FIG. 4D). Much higher amounts of HPCs were released from day 9 to day 15 and beyond as shown in FIGS. 4E and 4F. FIG. 4G, a higher magnification image, shows typical unattached round HPC morphology.

It is important to stress that this natural self-release of large quantity of HPCs in suspension is extremely beneficial for development of a harvesting process during large scale manufacture, which can be achieved through regular medium replenishment. HPCs in suspension can be easily harvested by volume reduction methods such as centrifugation or tangential flow filtration (TFF) devised for industrial scale production (Cunha et al. 2015).

### Histology and Immunofluorescence Analysis of HEC Markers in 3D Culture Spheres at Different Stage of Differentiation

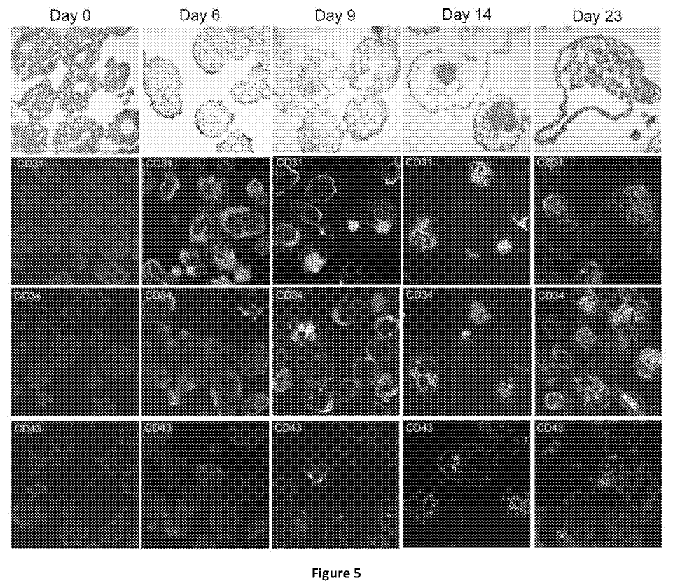

To visualize progressive morphological changes inside the cell spheres at different stages of hematopoietic differentiation, sections of spheres were either stained with hematoxylin (top row in FIG. 5) or with antibodies for CD31, CD34 and CD43 (lower 3 rows in FIG. 5). On day 0 with undifferentiated hiPSCs, cell spheres were more compact with most pronounced nuclear staining pattern, reflective of the large nuclear to cytoplasm ratio of typical pluripotent stem cells. No expression of CD31, CD34 and CD43 was found at Day 0. At the peak of HEC population at day 6, a clear transition from epithelial (day 0) to mesenchymal morphology was observed in all spheres. There is a strong CD31⁺ population inside all spheres, indicating highly efficient transition from hiPSCs to HECs. This is further confirmed by the presence of CD34⁺ cells as well as a small but distinctive number of CD43⁺ cells. Spheres on day 9 grew larger in size with formation of cavity inside. Expression of CD31 and CD34 remained high in the overall population. The relatively low percentage of CD43⁺ cells inside spheres indicated that most CD43⁺ cells were released into the media (FIG. 5). On day 14, much larger cavities in most spheres were present together with a core of more compact cells that were both CD31⁺ and CD34⁺. CD43⁺ HPCs were also present inside the spheres. The average spheres grew even bigger on day 23 of differentiation with a large cavity. CD34 expression remained very strong inside the cellular core of such spheres at this stage, indicating a robust long-term hematopoietic differentiation.

### Dynamic Change of Lineage Specific Marker at Different Stage of Differentiation

To define the best conditions to achieve optimal long-term hematopoietic differentiation efficiency, 3D sphere hematopoietic differentiation was tested under many different medium conditions (data not shown). Among all conditions tested, we identified the two best conditions (designated as Cond. A and Cond. B) suitable for this study.

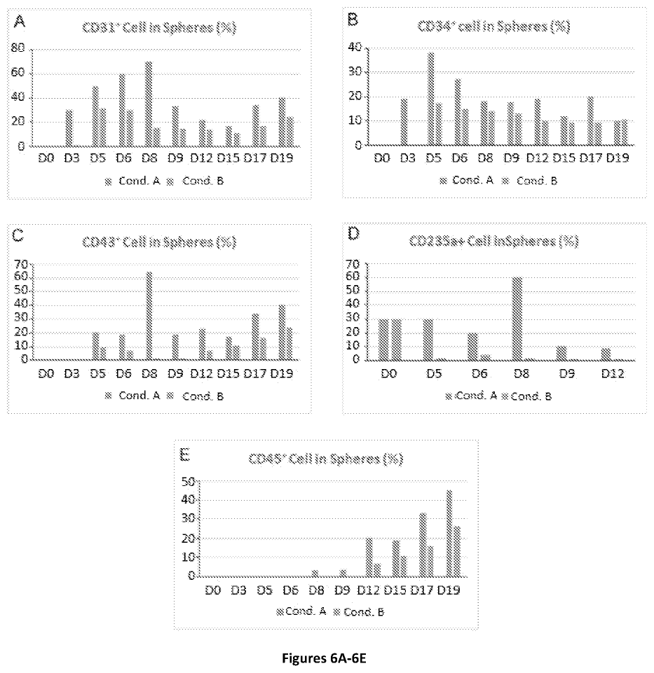

The starting hiPSC numbers for Cond. A and B experiments were identical at 20×10⁶ cells. From differentiation day 0 to Day 19, expression of lineage specific markers CD31, CD34, CD43, CD235a, and CD45 in cell spheres were analyzed by flow cytometry. As shown in FIGS. 6A-6E, significant variations in expression profiles were observed in all five markers between experimental cond. A and B. In Cond. A, percentages of CD31⁺, CD34⁺ and CD43⁺ cells in spheres were significantly higher than for Cond. B, confirming Cond. A is optimum for higher efficiency in HEC generation (FIGS. 6A-6C). The percentage of CD34⁺ and CD31⁺ in sphere cells of Cond. B was comparable to Cond. A in later stages of differentiation on day 19 (FIG. 6B). Expression of CD235a on progenitor cells specifies erythroid lineage potentials. The percentage of CD235a⁺ sphere cells was significantly higher in Cond. A and reached peak level at day 8. In contrast, the expression of CD235a was completely suppressed in sphere cells at day 5 of differentiation in Cond. B. During early hematopoiesis, previous reports have shown that suppression of CD235a expression in HECs through manipulating Wnt signaling pathways boosts definitive but suppresses primitive hematopoiesis (Sturgeon et al. 2014). The percentage of CD45⁺ cell in spheres were low until day 12 and increased significantly from day 12 to day 19 in both Cond. A and B. (FIG. 6E). Taken together, we conclude that Cond. A is the optimal condition for generating high percentage HECs in spheres. As shown in FIGS. 6A-6E, HPCs harvested early from spheres in Cond. A were suitable for generating erythrocytes and megakaryocytes. Alternatively, suppression of primitive hematopoiesis in Cond. B may drive early hematopoiesis in spheres toward definitive phenotype. Together with data shown in Tables 1A and 1B, spheres in Cond. B displayed much higher total cell counts and higher percentages of CD34⁺ cells and released more HPCs, particularly in later stages of differentiation. These observations strongly indicate that Cond. B is a better choice for producing definitive hematopoietic cells such as CD34⁺CD133⁺ hematopoietic stem cells (HSC). In conclusion, we have identified two conditions of 3D sphere hematopoietic differentiation, from which you can choose for different manufacturing purposes.

TABLE 1A
Estimated Sphere Cell Numbers (×10⁶)

| | Cond. A | Cond. B |
|---|---|---|
| Day 0 | 20 | 20 |
| Day 3 | 140 | 47 |
| Day 5 | 200 | 127 |
| Day 6 | 202 | 223 |
| Day 8 | 173 | 288 |
| Day 23 | 50.31* | 182.9* |

*Actual sphere cell counts are higher than this final harvest counts due to repeated sampling.

TABLE 1B
Sphere cell count and viability of CD34* and CD34 fractions at day 23 of differentiation

| | Cond. A | | Cond. B | |
|---|---|---|---|---|
| | Count (×10⁶) | Viability (%) | Count (×10⁶) | Viability (%) |
| CD34* | 4.83 | 85.00 | 40.4 | 79.3 |
| CD34 CD45* | 9.6 | 80.6 | 33.1 | 81.2 |
| CD34 DC45 | 35.88 | 88.2 | 109.4 | 82 |

| | | |
|---|---|---|
| Total count (×10⁶) | 50.31 | 182.9 |
| Percentage of CD34* | 9.60% | 22.09% |

### Release and Harvest of Large Quantity of HPCs

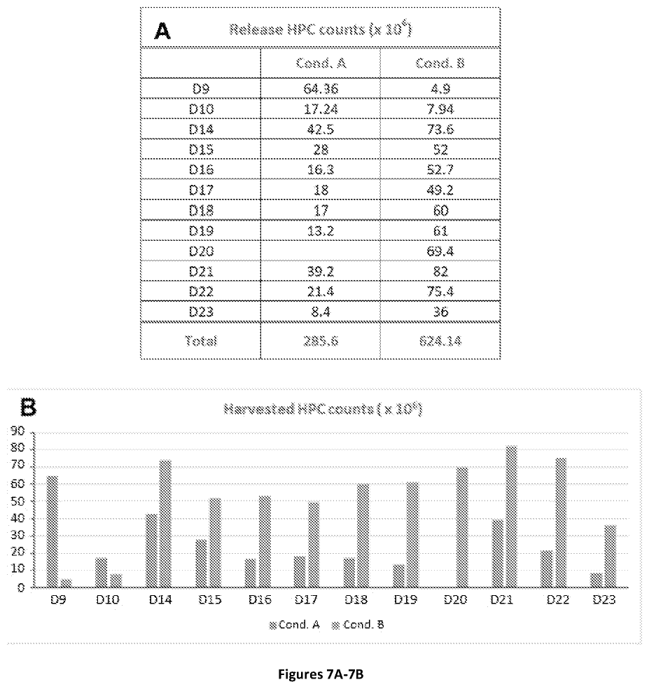

As shown in FIGS. 4A-4I, significant numbers of HPCs were released starting from day 8 to 9 of 3D sphere hematopoietic differentiation cultures. The number of released HPCs was steadily increased from day 9 onward. HPCs were collected either daily or every other day from experimental Cond. A and B, and the total cell numbers for each collection were shown in FIGS. 7A and 7B. In Cond. A, the combined total harvest of HPCs was 285.6×10⁶; whereas the combined total harvest of HPCs reached 624.14×10⁶ for Cond. B. On both days 9 and 10, spheres in Cond. A released more HPCs than did the spheres in Cond. B. From days 14 to 23, however, spheres in Cond. B released significantly more HPCs than spheres in Cond. A. This reverse trend of HPC release from spheres is consistent with the hematopoietic lineage marker expression profile (CD31, CD34, CD43, CD235a, CD41 and CD45) of sphere cells shown in FIGS. 5 and 6A-6E, suggesting a distinct preference of definitive versus primitive hematopoiesis under the two conditions. Our results on both sphere cells as well as released HPCs clearly demonstrate that we have successfully developed a highly efficient 3D hematopoietic differentiation process. Under optimized conditions, each input hiPSC can generate up to 31 HPCs in our current protocol. A 1000 ml bioreactor will be able to accommodate 600-1000×10⁶ undifferentiated hiPSCs, the predicted final HPC output for a 25-day production process could reach 3.1×10¹⁰ cells.

Characterization of harvested HPCs

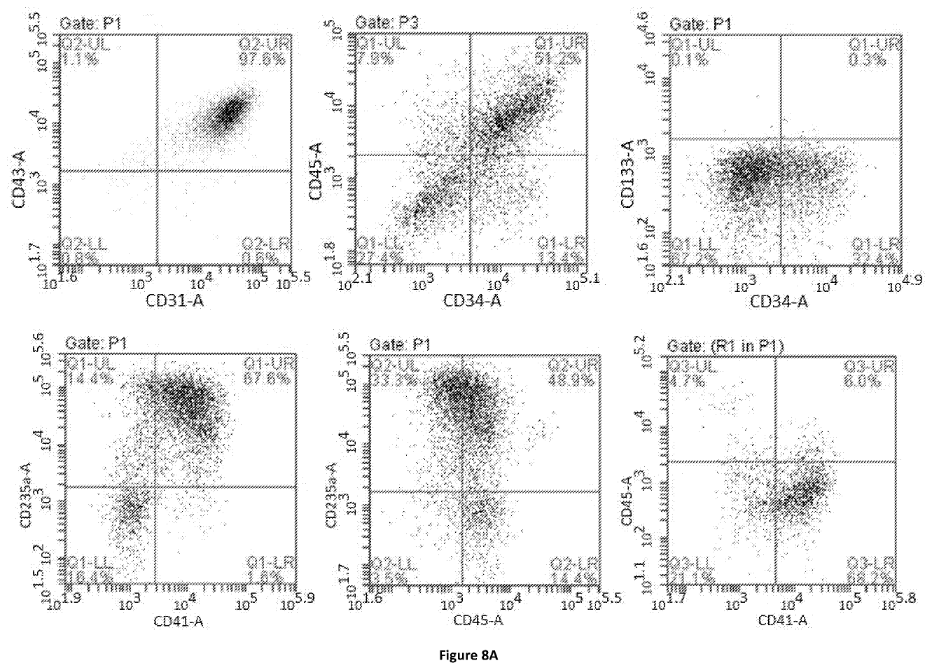

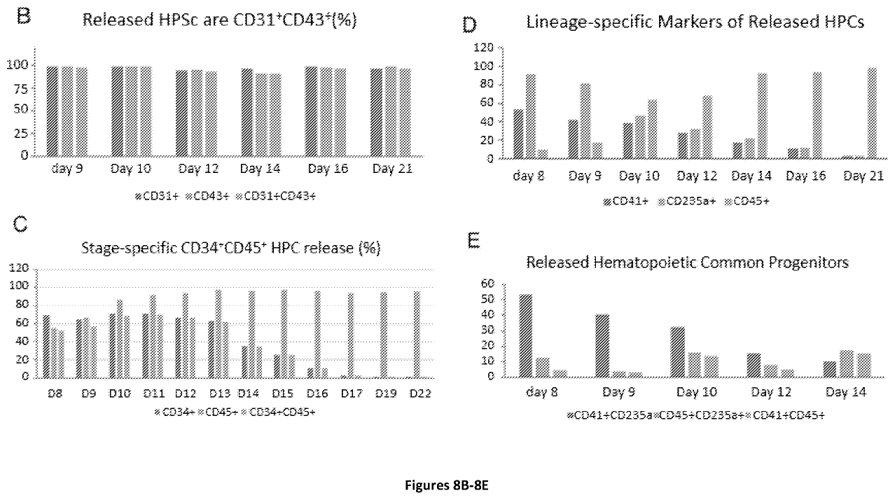

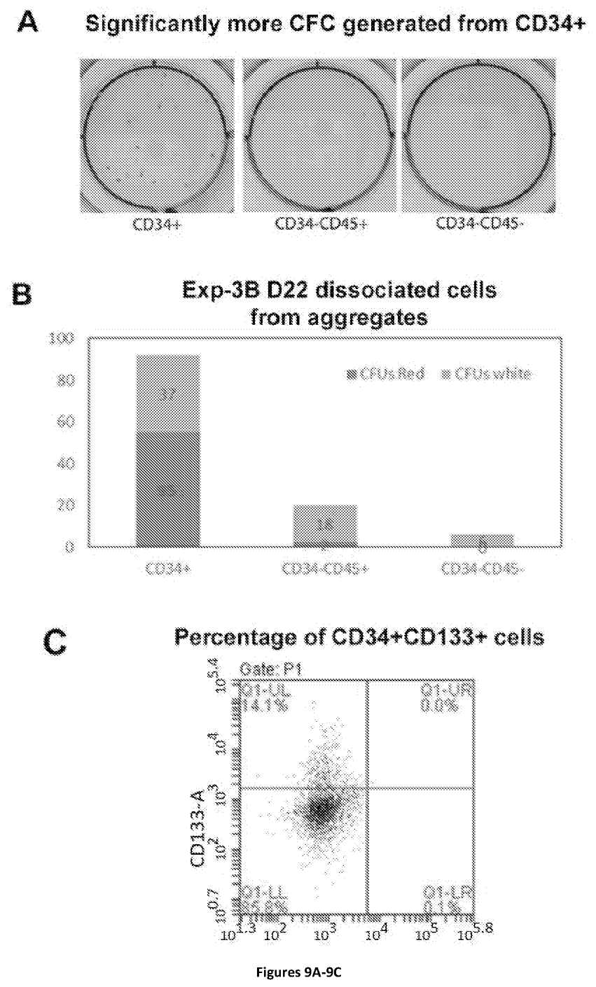

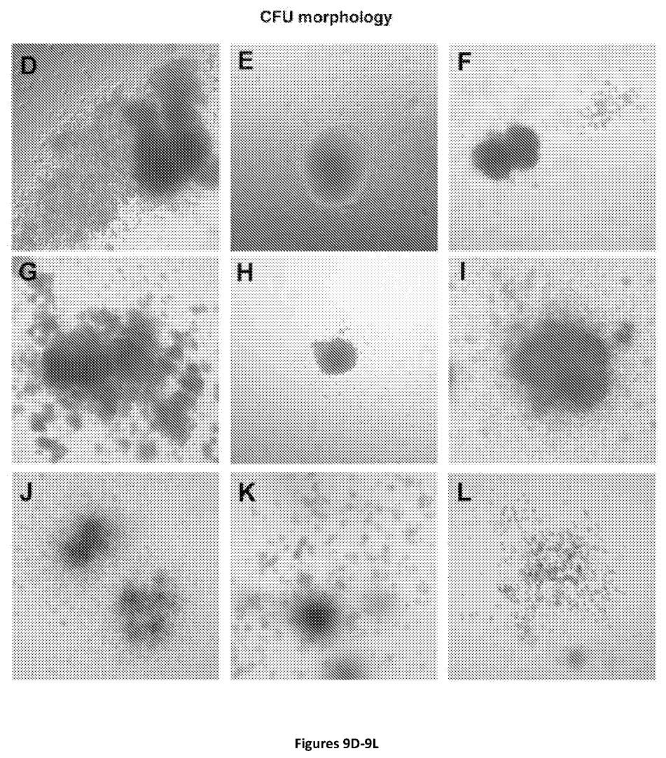

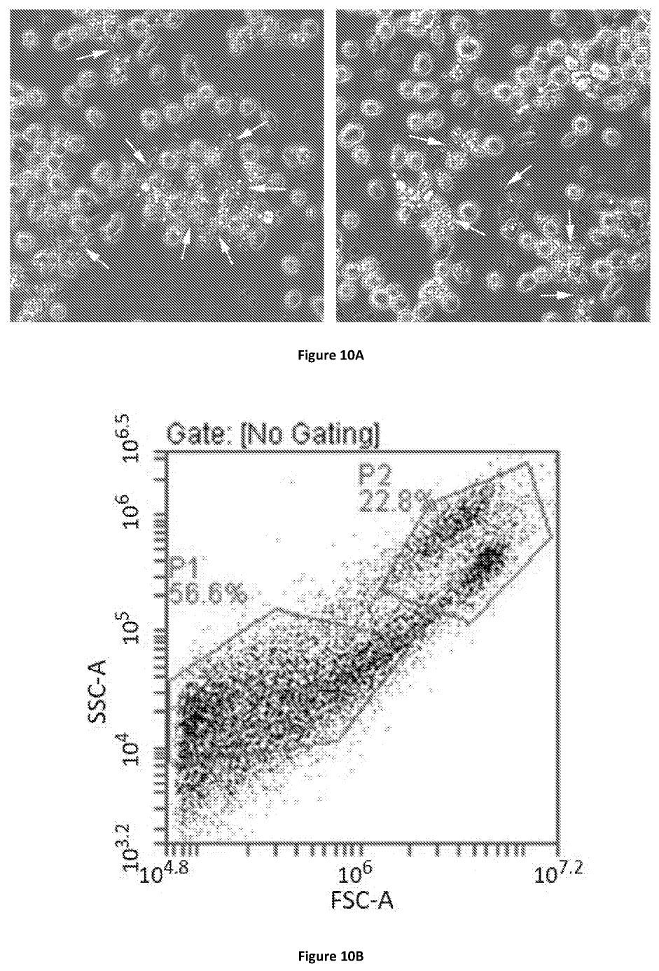

Hematopoietic lineage specific marker expression of harvested HPCs were analyzed by flow cytometry. As shown in FIG. 8A, HPCs harvested from a representative experiment on Day 9 were 97.6% CD31⁺CD43⁺, indicative of their HEC origin as well as full commitment to hematopoietic lineage. There was also a strong presence of CD34⁺CD45⁺ HPCs, but not CD133⁺ HPCs at this stage. A high percentage (68%) of HPCs were CD41⁺, indicating predominantly megakaryocyte lineage potential as reported previously (Feng et al. 2014). A majority of HPCs were either common progenitors of megakaryocyte/erythroid lineage (CD41⁺CD235a⁺) or common progenitors of erythroid/myeloid lineage (CD45⁺CD235a⁺, only very few of these cells were megakaryocyte/myeloid common progenitors (CD41⁺CD45⁺).

As shown in FIG. 8B, HPCs collected at various stage of differentiation were all CD31⁺CD43⁺ confirming their high purity. CD34⁺CD45⁺ HPCs are thought to possess multi-lineage potential capable of generating not only myeloid but lymphoid lineage cells such as NK cells (Knorr et al. 2013). In one representative experiment shown in FIG. 8C, expression of both CD34 and CD45 on HPCs was tracked daily from day 8 to day 17, and a significant percentage (>60%) of the released HPC population from day 8 to day 13 was CD34⁺CD45⁺, then these cells decreased gradually from day 14 (34%) to day 17 (2%).

As shown in FIG. 8D, early (day 8 and day 9) HPCs were predominantly CD41⁺ and CD235a⁺, however, the HPC population was gradually replaced by CD45⁺ HPCs. Similarly, the percentage of CD41⁺CD235⁺ MK/erythroid common progenitors were highest on day 8 and gradually decreased from day 9 to day 14. Interestingly, other common progenitors such as CD45⁺CD235a⁺ and CD41⁺CD45⁺ HPCs were also observed from day 10 to day 14.

Our results demonstrate that our new process can generate large quantity of variable hematopoietic progenitors that are suitable for future manufacture of cells of both lymphoid (NK or T cells) or myeloid (macrophages, neutrophils, etc.). These cells are key components of new generation of immune-therapies such as CAR-NK and CAR-macrophages.

### Isolation, Characterization of CD34⁺ Hematopoietic Stem Cells in 3D Spheres

The release of large quantities of HPCs from spheres into medium in our system clearly indicates strong active and dynamic hematopoiesis inside the 3D sphere structures. We therefore speculate that multipotent hematopoietic stem cells (HSCs) may be generated inside these spheres. At various days of differentiation, cell spheres were dissociated into single cells and CD34⁺ and other cells were analyzed. As shown in Table 1A, significant cell expansion was observed in both Cond. A and B. Starting from 20×10⁶ hiPSCs on day 0, 173×10⁶ (Cond. A) and 288×10⁶ (Cond B) sphere cells were obtained on day 8, and 50×10⁶ (Cond. A) and 183×10⁶ (Cond. B) cells at day 23, respectively. Among these cells, about 10% from Cond. A and 22% from Cond. B were CD34+ hematopoietic stem cells. Since significant numbers of spheres were removed during the whole process for various analyses, the actual cell numbers harvested from dissociated spheres should be significantly higher. These results demonstrate that this new 3D sphere environment is adequate to support healthy long-term growth and differentiation of hematopoietic cells.

To quantitatively evaluate the hematopoietic lineage potential of CD34⁺ cells, dissociated single cells from Cond. A and Cond. B on Day 22 were separated into CD34⁺ and CD34⁻ populations. The CD34⁻ fraction was further separated into CD34⁻CD45⁺ and CD34⁻CD45⁻ populations. As shown in Table 1B, dissociated sphere cells remained viable after extended dissociation process. A higher yield of the CD34⁺ population was achieved from Cond. B, which also produced the highest numbers of released HPCs (see FIGS. 7A-7B).

In contrast to CD34⁻ fractions, cells of the CD34⁺ fraction showed increased colony forming capability (FIGS. 9A and 9B). Flow cytometer analysis of CD34⁺ fraction demonstrated that 14% of the population were also CD133⁺ (FIG. 9C), confirming the existence of CD34⁺CD133⁺ engraftable HSC subpopulation (Drake et al. 2011). As shown in FIGS. 9D-9I, significant numbers of red or mixed red (FIGS. 9G and 9I) colonies of both BFU-E (FIG. 9D) and CFU-E (FIGS. 9E, 9F and 9H) were generated from CD34⁺ cells. Colonies of myeloid lineages such as CFU-G (FIG. 9J), CFU-M (FIGS. 9K and 9L) were also observed. Many big mixed red colonies in CFU cultures strongly indicates the presence of HSCs inside the differentiated spheres at later stages of differentiation. Long term CD34⁺ cells that are capable of long term engraftment in humanized mice can also be generated using the methods disclosed herein.

### Example 2: Production and Characterization of Specific Hematopoietic Lineages

In vitro differentiation of NK as well as other cells of lymphoid lineages has been shown to require co-culture with feeder cells over-expressing Notch signaling ligand DLL-1/4 as previously reported (Watarai et al. 2010; Zeng et al. 2017; Ditadi et al. 2015). Here we present a novel scalable 3D system to robustly generate almost a pure population of NK cells from human PSCs under defined serum-free and feeder-free conditions. Our discovery represents a breakthrough technology in the development of large scale manufacture of not only NK cells, but other cell types of lymphoid and hematopoietic lineages as well. Furthermore, as demonstrated herein, our 3D hematopoietic differentiation system is different from all available pluripotent stem cells (PSC) differentiation methods and is suitable for industrial scale manufacture for off-the-shelf immune cell products such as NK and T cells for immune oncology therapies.

Platelet and RBC Formation from Hematopoietic Progenitors

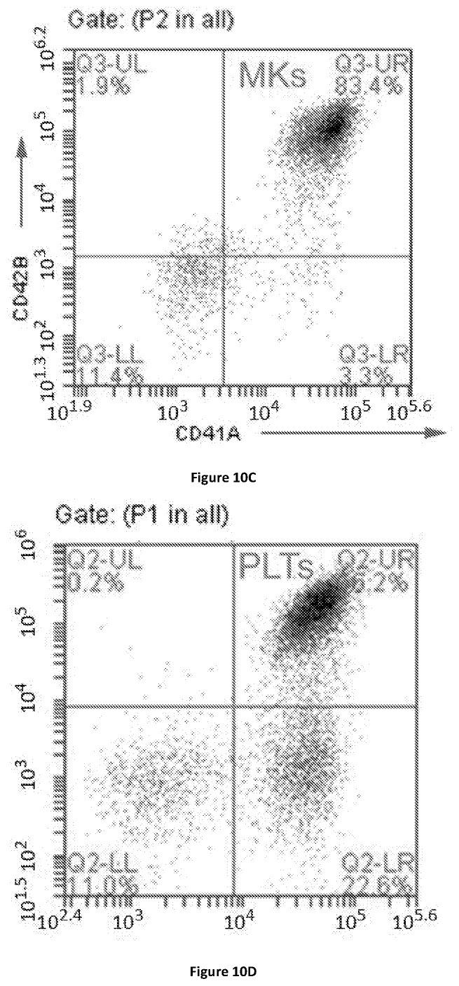

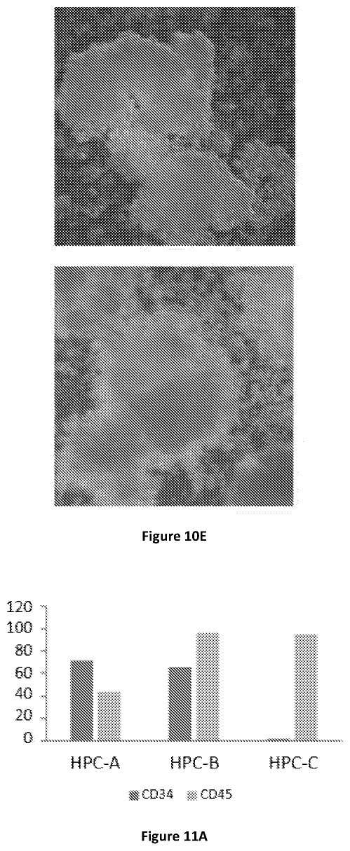

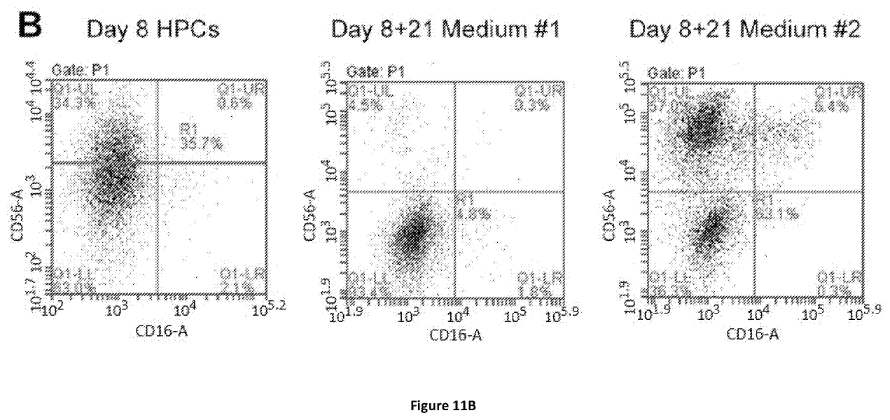

One important potential application of harvested HPCs is for large scale manufacture of megakaryocytes and platelets as reported previously (Feng et al. 2014; Thon et al. 2014). HPCs harvested on day 8-10 were cultured in MK promoting medium as published earlier (Feng et al. 2014) for 5-7 days. As shown in FIG. 10A, significant formation of proplatelets (pointed by white arrows) was observed after 3 days of incubation in MK promoting medium. Platelets in the MK medium were harvested as described earlier (Feng et al. 2014) and analyzed for expression of MK-specific CD41a and CD42b on both platelets (as shown in Gate P1 in FIG. 10B) and MKs (Gate P2 in FIG. 10B). The percentage of CD41a⁺CD42b⁺ megakaryocytes reached 83.4%, and 66.2% of CD41⁺CD42⁺ platelets were also obtained (FIGS. 10C and 10D). It was confirmed by an earlier report that platelets derived in similar fashion in 2D culture systems were fully functional and displayed similar ultrastructural morphology with human platelets in circulation (Feng et al. 2014). MKs derived from our 3D sphere system display equivalent characteristics. In conclusion, generation of megakaryocytes and platelets under complete 3D culture system has major advantages over 2D system reported by us and many other labs, not only in scalability but also functional relevance due to constant presence of shear force mimicking in vivo circulation.

As shown in FIG. 8, early HPCs harvested from Day 8-10 were mainly CD235a⁺, indicating their erythroid lineage. We observed formation of very large CFU-e colonies when these CD235a⁺ HPCs were plated in CFU-forming medium (FIG. 10E), which suggests CD235a⁺ HPCs are suitable for large scale manufacture of designer RBCs that can either be used for blood transfusion or as targeted drug carrier (as new technology currently in development by Rubius Therapeutics, Cambridge, Mass.).

Derivation and Characterization of CD56⁺ NK from Early HPCs

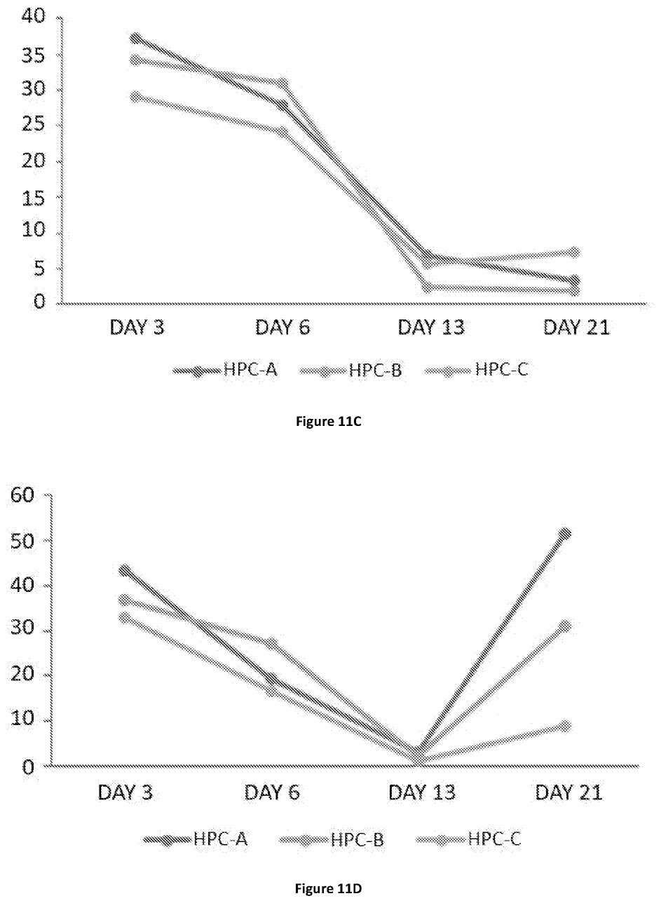

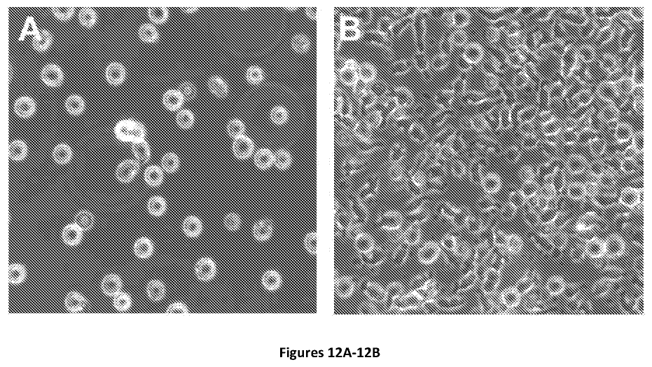

NK cells could play very important roles in the next generation of cancer immunotherapies. Currently, it is technically challenging to obtain large quantity of NK cells through amplification from autologously harvested peripheral blood cells. We demonstrated here that hematopoietic progenitors generated in our 3D differentiation system can be efficiently differentiated into NK cells. HPCs harvested from day 8 (designated as HPC-A), day 11(HPC-B) and day 18 (HPC-C) were cultured in 2 media (#1 and #2) formulated for NK cell differentiation and maturation for additional 21 days. As shown in FIG. 11A, these HPCs harvested at different times showed distinct hematopoietic surface marker profiles: approximately 60-70% and 40% of HPCs-A expressed CD34 and CD45, respectively; the expression of CD34 remained similar, but almost 100% of HPCs-B were positive for CD45; CD34 expression was barely detectable in HPCs-C, while 100% of them expressed CD45, indicating maturation toward hematopoietic cells. We also observed that about 30% of all three HPC populations collected at different times expressed low levels of CD56, which is consistent with results shown in FIG. 8C. After being cultured in both media, CD56^{low} cells were gradually lost from days 6 to 13 for all three HPC collections. Furthermore, no CD56⁺ cells emerged from HPC-A, HPC-B and HPC-C in medium 1 at days 21, (FIG. 11C). In contrast, significant numbers of CD56^{high} cells re-emerged in medium #2 after culturing for 21 days, especially HPCs-A, from which a distinct cell population of CD56^{high} was observed (FIG. 11D). This re-emerged CD56⁺ population expressed higher level of CD56 than their HPC precursors (FIG. 11B, Day HPCs vs Day 8+21 Medium #2), indicating generation of CD56^{+high} cells with NK lineage.

Integration of 3D Spheres with 3D Scaffolds for Generation of Pure NK Cells Under Serum- and Feeder-Free Condition

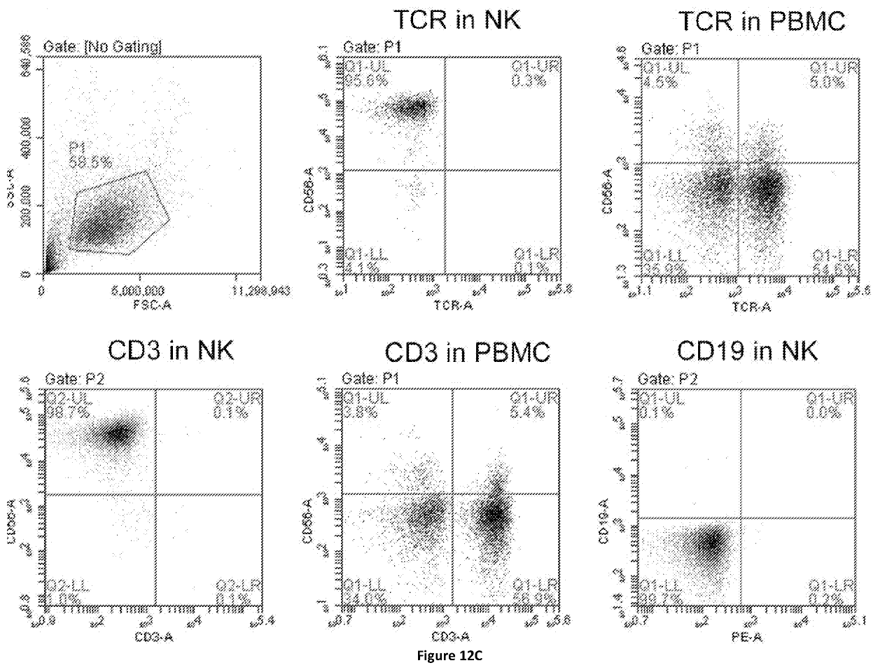

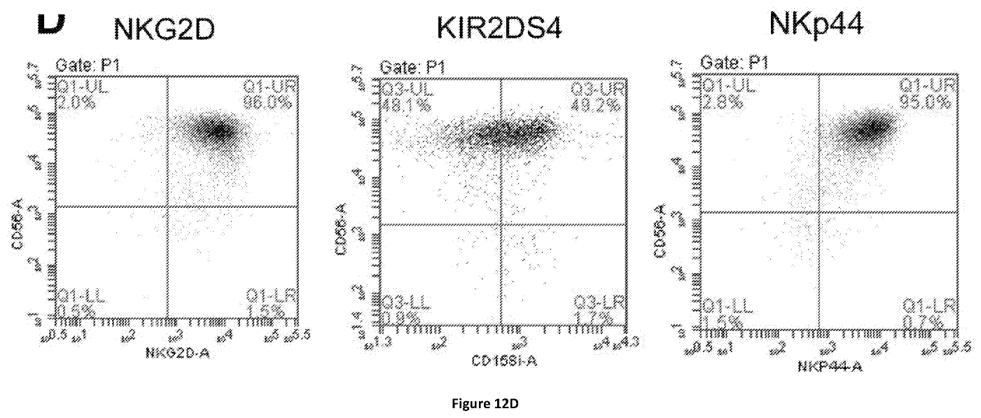

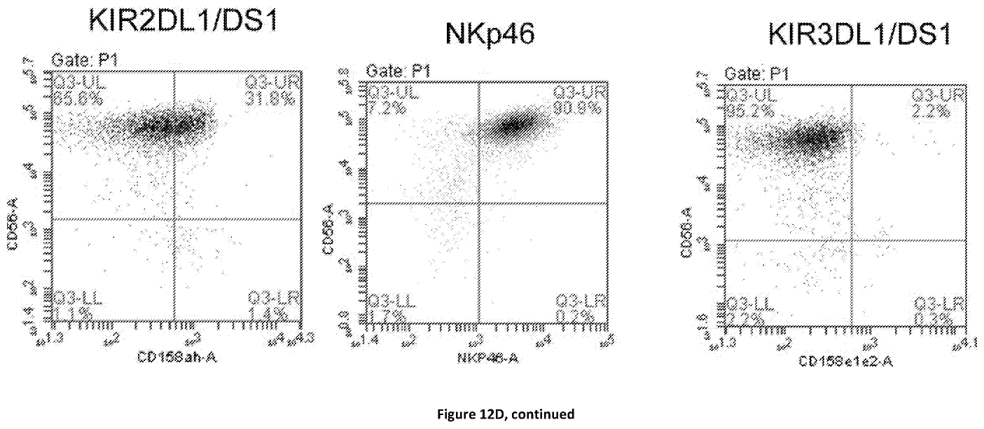

A previous study suggests a 3D architecture of the thymus provides optimal environment for T lymphocyte development (Mohtashami and Zuniga-Pflucker 2006). To improve NK differentiation and generation under serum-free and feeder-free conditions, Day 6 HECs were seeded into 3D scaffolds mimicking the in vivo niche to promote NK specification. Excellent HEC growth and differentiation were observed inside the scaffolds and large numbers of cells were released from Day 16. Approximately 10×10⁶ cells were collected from initially seeded 2×10⁶ HECs in a period of 10 days. As shown in FIGS. 12A-12D, cells released from scaffolds displayed a very distinct morphology from typical round-shaped HPCs (FIGS. 12A and 12B). Forward and side scattering plots of flow cytometry analyses shows that the released cells are highly homogeneous (FIG. 12C, top left). Over 96% of these cells were CD56^{+high} (FIGS. 12C and 12D), indicating a pure NK population. Unlike T lymphocytes in PBMC (top right), the released CD56⁺ NK cells did not express T-cell receptors (TCRs) (FIG. 12C, top middle), neither did they express the pan T-cell marker CD3 (FIG. 12C, lower left), while a significant fraction of PBMCs expressed CD3 antigen (lower middle). Additionally, B-cell marker CD19 was not detected in hiPSC derived-NK cells (FIG. 12C, lower right). NKG2D is a transmembrane protein that belongs to the CD94/NKG2 family of C-type lectin-like receptors expressed on human NK cells (Houchins et al. 1991). NKp44 (Vitale et al. 1998) and NKp46 (Sivori et al. 1997) are NK-specific surface molecules involved in triggering NK activity in human. We demonstrated that hiPSC-CD56^{+high} cells were NKD2G⁺ (96%), NKp44⁺ (95%) and NKP46⁺ (90.9%) (FIG. 12D, left panel). Killer-cell immunoglobulin-like receptors (KIRs), a family of type I transmembrane glycoproteins, are expressed on the plasma membrane of NK cells and a minority of T cells (Yawata et al. 2002; Bashirova et al. 2006). They regulate the killing function of these cells by interacting with major histocompatibility (MHC) class I molecules. Various percentages of the CD56⁺ cells were KIR2DS4⁺ (49.2%) and KIR2DL1/DS1⁺ (31.8%), almost all these CD56⁺ cells were KIR3DL1/DS1⁻ (97%, FIG. 12D, right panel), indicating their diversity in KIRs types of hiPSC-NK populations generated in our 3D system. These observations demonstrate that these CD56^{+high} cells are bona fide NK cells.

Cytotoxic Activity of iPS-NK on K562 Target Cells

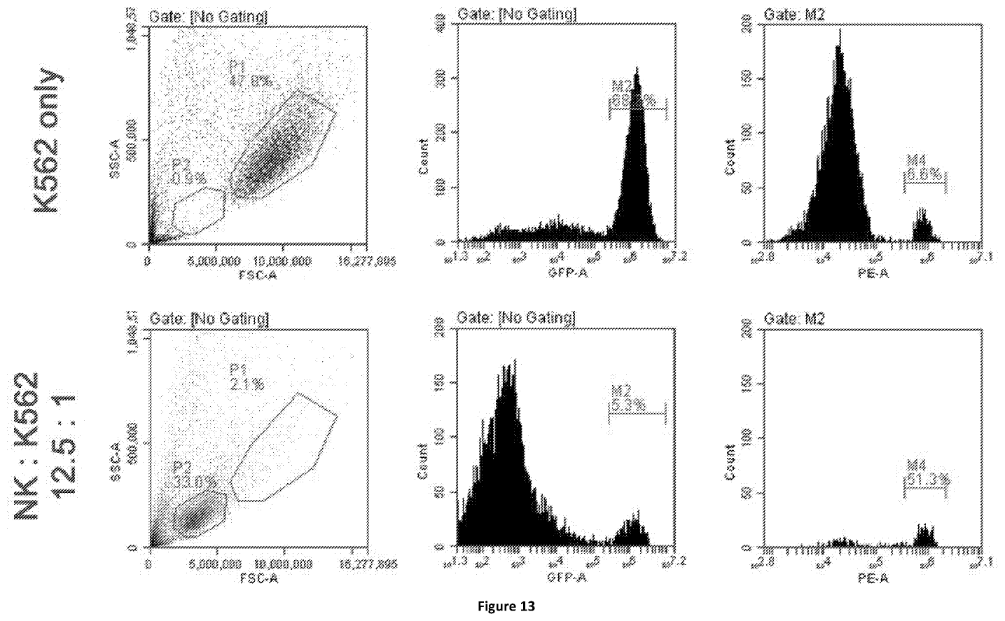

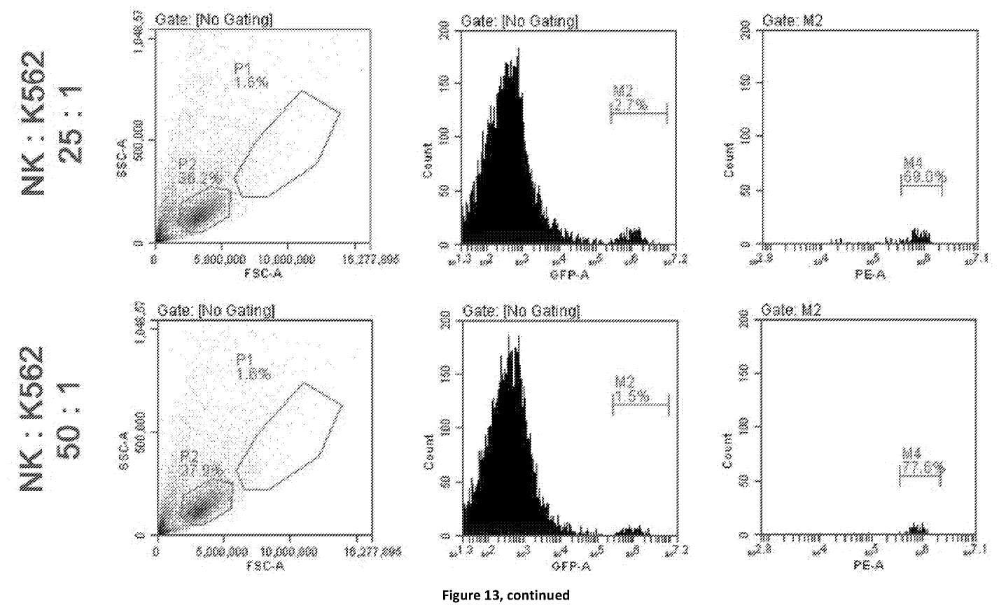

As shown from the left column in FIG. 13, NK effector cells (P2) have a very different forward/side scattering profile than target K562 cells. K562 cells are GFP⁺ while iPS-NK cells are GFP⁻ (shown in middle column). After a 2 hour incubation with effector NK cells, almost all target GFP⁺ K562 cells were destroyed by the iPS-NK cells regardless of the E:T ratio as shown from second to bottom row Small amounts of remaining K562 cells are mostly non-viable as shown in left column. This result confirms the iPS-NK cells we generated from this new technology platform not only share all cellular markers of NK cells, but also can kill potential target cells with deadly efficiency.

RNAseq Analyses Confirms that Human iPS-NK Cells are Authentic NK Cells

Summary: By comparative RNAseq analysis, human iPS-NK cells were compared with primary human NK cells and the results confirm that human iPS-NK cells assembled to human primary NK cells.

To investigate whether iPS-NK cells are true human NK cells, RNA-seq expression profiles of human iPS-NK cells were compared to two publicly available high-quality RNAseq data sets with different types of human immune cells. Dataset 1 (Racle et al. 2017) comprises reference gene expression profiles of sorted immune cells from human blood built from three studies (Racle et al. 2017), comprising of B cells, CD4, CD8, monocytes, neutrophils and NK cells. Dataset 2 (Calderon et al., available at www.ncbi.nlm.nih.gov/geo/query/acc.cgi?acc=GSE118165) comprises reference gene expression profiles of sorted immune cells with 166 human samples of 25 blood cell types from 8 health donors (Calderon et al., available at www.ncbi.nlm.nih.gov/geo/query/acc.cgi?acc=GSE118165). Human iPS-NK cell raw counts (iPS-NK3, iPS-NK8 and iPS-NK12) were transformed to TPM (transcript per million reads) based on human genome version h19. Expression profiles were combined between human iPS-NK data and reference data based on matched unique gene symbols and normalized by total intensity across all samples. Cell markers for different types of immune cells were from Racle et al. The 1000 most variable genes in the reference dataset was used to calculate the similarity of any two samples by Pearson correlation. Heatmap of gene expression profiles and correlation were visualized with TMev.

Based on expression analysis of specific cell markers and similarities in global expression profiles, human iPS-NK cells assembled to human primary NK cells. (Dataset 1: Average Correlation of human iPS-NK to self: 0.89, to primary NK cells: 0.53, to other cell types: 0.31; Dataset 2: Av. Correlation of human iPS-NK to self: 0.891, to naïve NK: 0.283, to activated NK:0.229, to other cell types: 0.082). However, three batches of human iPS-NK cells showed some variations, and iPS-NK3 sample (about 95% CD56+ cells) closely matches primary human NK cells, while samples of iPS-NK8 and iPS-NK12 expressed some markers of macrophages and monocytes compared to both reference datasets, such as typic markers of CD14, CD33, and CSF1R. Theses macrophage/monocytic features are consistent with the purities of these two batches of human iPS-NK cells (87% and 75% CD56+ for iPS-NK8 and iPS-NK12 samples, respectively). In summary, these results are very consistent based on comparative analysis with two different public RNAseq data sets, and confirm that human iPS-NK cells are authentic NK cells.

High Percentage (>80%) Human iPS-NK Cells are CD56+CD8+ Effector Cells

Summary: We unexpectedly discovered that over 80% of human iPS-NK cells generated using our technology platform are CD56⁺CD8⁺, indicating the strong presence of cytotoxic effector cells.

Different subsets of NK cells have been described in human peripheral blood. The majority of peripheral blood NK cells are CD56dimCD16+ cells, whereas lymph node resident NK cells are predominantly CD56brightCD16-NK cells (Ahmad et al. 2014). Using our 3D in vitro human iPS differentiation system, we discovered that human iPS-NK cells are over 95% CD56brightCD16-. These results suggest that our hematopoietic cellular spheres likely resemble lymph node tissue in vivo providing ideal niche environment for NK cell differentiation and development.

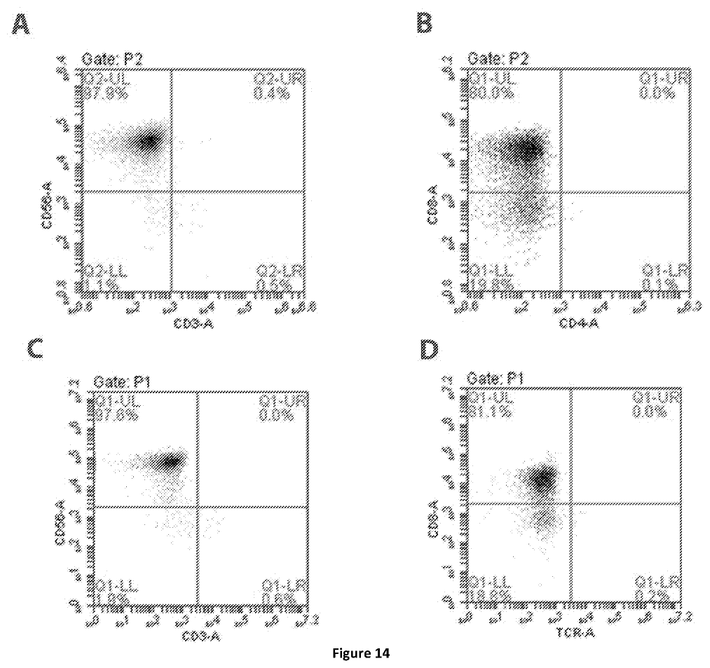

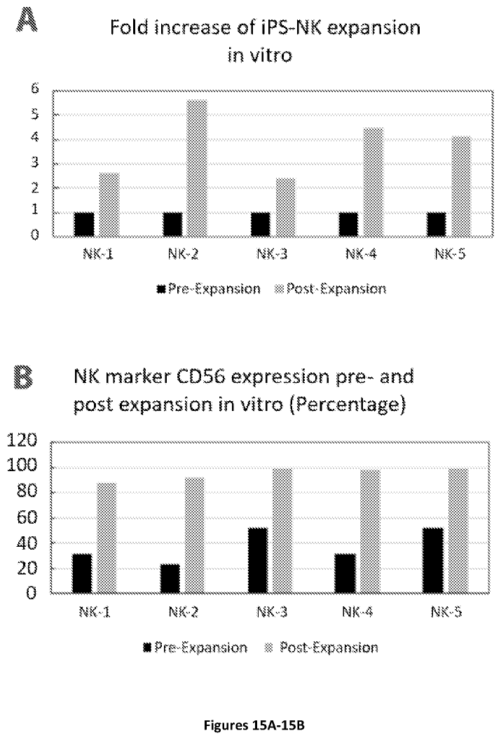

Roughly 30% of human peripheral blood NK cells express the CD8 marker (Ahmad et al. 2014; Addison et al. 2005). As shown in FIG. 14, it was surprisingly discovered that over 80% of human iPS-NK cells derived by our 3D HSC differentiation system are CD56+CD8+. It has been confirmed by previous report that CD56+CD8+ human NK cells display higher cytolytic function than CD56+CD8-subset NK cells (Addison et al. 2005). High frequency of CD8+NK cells are associated with slower disease progression of HIV infection (Ahmad et al. 2014; Rutjens et al. 2010). These results demonstrate that our 3D differentiation platform preferentially generate highly cytotoxic CD56+CD8+ subset NK cells. Adoptive transfer of predominantly CD56+CD8+NK cells may translate into better clinical outcome for anti-cancer or anti-viral infection therapies.

In Vitro Expansion Under Feeder-Free Conditions Results in High Yield and Purity of Human iPS-NK Cells

Summary: In order to improve the yield and purity of iPS-NK cells harvested from bioreactors, we have demonstrated that harvested NK can be further expanded and enriched via a feeder-free defined culture medium.

Due to lack of sufficient NK cells from peripheral or cord blood, donor-sourced NK cells need to be expanded in order to generate therapeutic doses of human NK cells for cell therapy. Efficient expansion of donor NK cells is dependent on presence of feeder cells such as artificial antigen presenting cells (iAPCs). Due to low NK lineage specific differentiation under 2D conditions, previously reported human iPS-derived NK cells also require feeder-dependent expansion (Li et al, 2018). The use of modified cancer feeder cells is not only cumbersome but also carries the risk of contamination with unwanted cells in the NK cell population.

In addition to the superior scalability of the 3D bioreactor human iPS-NK differentiation and production system described herein, feeder-free expansion of human iPS-NK cells was also investigated. The results, as shown in FIGS. 15A-15D, demonstrate that 5 different batches of human iPS-NK cells harvested at various stages of differentiation expanded about 3- to 5-fold using the presently described feeder-free expansion system. More importantly, this system not only expands these cells but also enriches the CD56+NK cell population. Less than 40% of the CD56+ population was enriched to reach>95% CD56+ cells after one to two weeks of expansion. These data demonstrate that human iPS-NK cells/progenitors from different differentiation stages can be further expanded under feeder-free condition, resulted in significantly higher purity of CD56+NK cells.

CD3+T Lymphocyte Generation from 3D Hematopoietic Differentiation Platform

Summary: In addition to human iPS-NK cells, we have demonstrated that our system can be used to efficiently generate CD3⁺ iPS-T cells, which strongly indicates that we have successfully recreated long lasting hematopoiesis niche environment with definitive phenotype in our 3D sphere culture system.

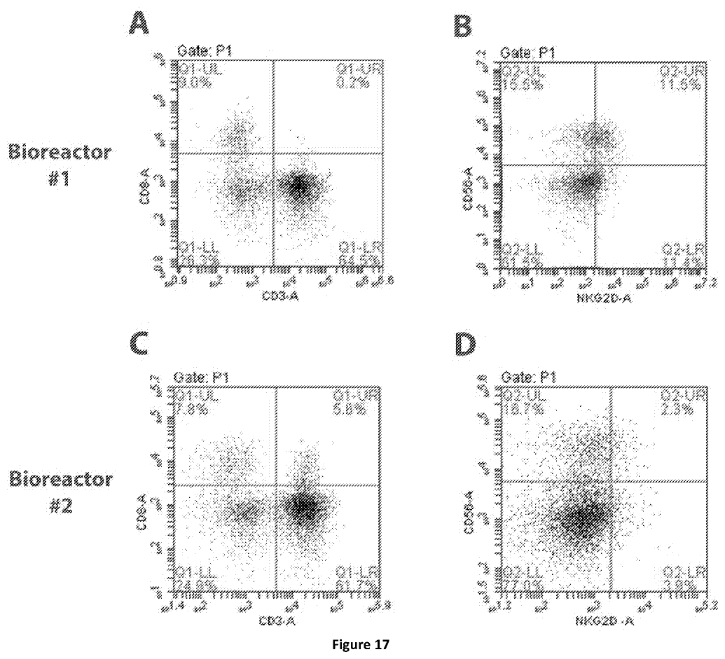

Lineage specific differentiation of T lymphocytes is technically challenging. Most previous reports of T lymphocyte differentiation from hES/iPS cells were using feeder-dependent methods. Developing a scalable 3D bioreactor system to generate pure T lymphocytes at an industrial scale is highly attractive for future immune-oncology therapies. Using the same platform system for the generation of iPS-NK cells with some modifications, relatively pure (>60%) CD3 T lymphocyte-like progenitors were generated (FIG. 17) in two separate experiments. These results are significant for the following reasons: (1) both CD3-NK cells and CD3+ T cells may come from the same common lymphoid progenitors; (2) these common lymphoid progenitors are efficiently generated in spheres undergoing hematopoietic differentiation in our 3D differentiation system; and (3) hematopoiesis within these late stage spheres are of definitive phenotype. Further optimization of the 3D sphere differentiation system favoring T lymphocyte lineage will significantly improve yield, purity, and functionality of iPS-T cells. These results further confirm the initial claim that this 3D hematopoietic differentiation system is a versatile platform technology that can be adapted to manufacture all hematopoietic lineage cells including hematopoietic stem cells.

Human iPS-NK Selectively Kill K562 Cancer Cells but not Normal Cells

Summary: Additional cytotoxic analysis of human iPS-NK cells against both normal and cancer cells confirm that human iPS-NK cells selectively kill cancer cells but not normal cells.

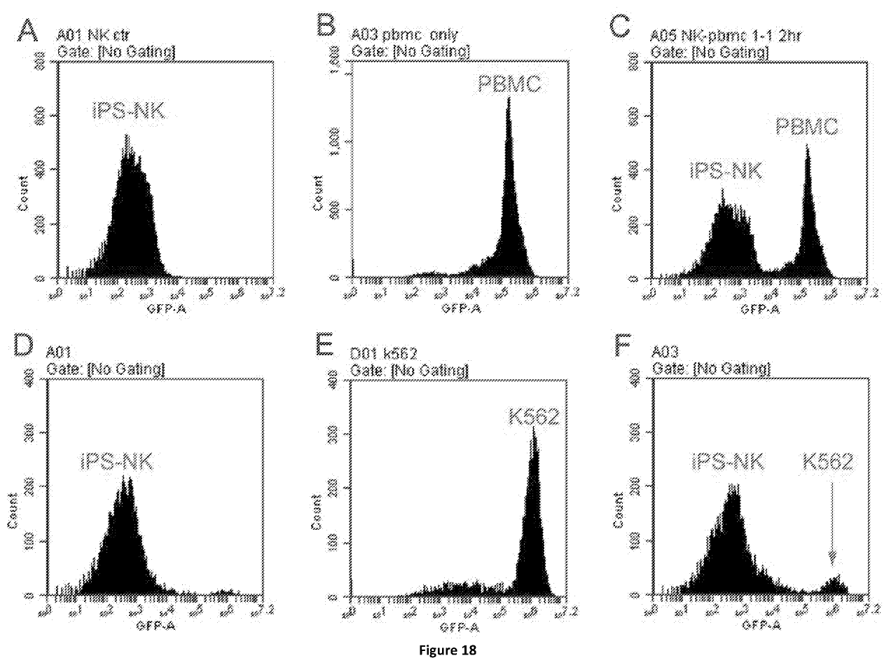

Strong cytotoxic activity against K562 cancer cells was demonstrated above. A similar anti-cancer cytotoxic effect was observed with OCI-AML3 and GMB leukemic cells and BxPC-3 pancreatic cancer cells. To confirm that human iPS-NK cells with strong cytotoxic activity can distinguish between normal and cancer cells, fluorescence labelled normal human peripheral blood mononucleotide cells (PBMC) and K562 cells were mixed with human iPS-NK cells at 1:1 ratio and incubated for 2 hours. As shown in FIG. 18, more than 80% of K562 cells were killed, whereas no obvious cytotoxic activity towards normal human PBMC was observed, demonstrating the cytotoxic specificity of human iPS-NK cells toward abnormal (cancer) cells, but not normal cells.

### Example 3: Recapitulation of NK Lineage Specific Differentiation in 500 ml Bioreactor

Summary: To confirm that our 3D suspension culture system can be scaled up to meet industrial demand, we also demonstrated that human iPS-NK lineage specific differentiation in smaller 30 mL bioreactors can be replicated in 500 mL bioreactor.

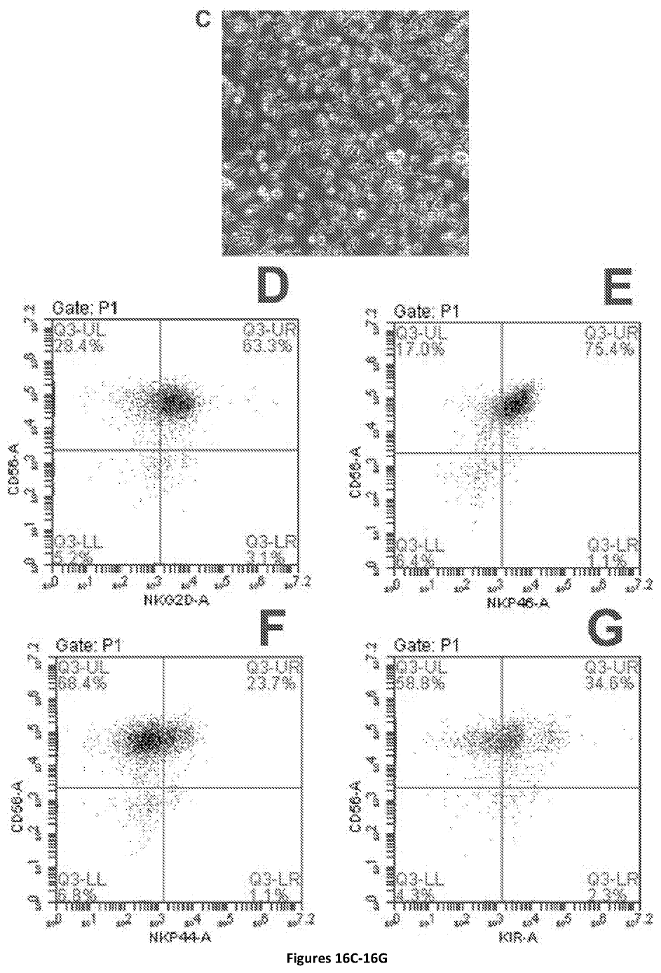

One of the major strengths for the presently disclosed 3D differentiation system is its scalability. To verify whether lineage specific differentiation can be recapitulated in a large volume bioreactor, parallel NK lineage specific differentiations were performed in both small 30 ml and large 500 ml bioreactors using identical iPS cells. To confirm induction of hemogenic endothelial (HE) lineage at early phase, HE markers CD31, CD144 (VE-Cad), and CD34 and hematopoietic marker CD43 were analyzed in spheres at Day 3 and Day 5 of differentiation. As shown in FIG. 16A, although CD31 and CD144 expression was higher in spheres from 30 ml bioreactors than those from 500 ml bioreactors on Day 3, both markers reached similar levels (60-70%) on Day 5. Expression of CD34 and CD43 in spheres from 30 ml and 500 ml bioreactors was very similar on Day 3 and Day 5. The data confirm that induction of hemogenic endothelial lineage in 500 ml bioreactors is almost identical to that in 30 ml bioreactors.

The kinetics of CD56+NK cell generation from one 500 ml bioreactor was compared with results from 3 individual 30 mL bioreactors. As shown in FIG. 16B, the emergence of CD56+NK cells in the 500-mL bioreactor (shown in solid line) is highly comparable to that in all three 30 ml bioreactors (>90% cells are CD56+ at Day 46). Cells harvested on Day 46 show homogeneous iPS-NK morphology (FIG. 16C), and the majority of these cells also express NK cell-specific activating receptors NKG2D and NKp46. About 25% and 35% of these cells are positive for activating receptor NKP44 and inhibitory receptor KIRs, respectively (FIGS. 16D-16G). These results demonstrate that the NK lineage specific differentiation process can be replicated in larger bioreactors. Further scaled-up production of iPS-NK cells using a bioreactor larger than 500 mL, e.g., 1 liter, 10 liters, 100 liters, etc., is reasonably expected to be also feasible and practical.

### Example 4: Methods and Materials

### Cell Lines and Reagents

Four human induced pluripotent stem cell (hiPSC) lines used in this study were generated from human normal dermal fibroblast (hNDF) cells by using the StemRNA™-NM Reprogramming kit (Stemgent, Cat #00-0076). HiPSCs were grown in vitro as colonies on 0.25 μg/cm² iMatrix-511 Stem Cell Culture Substrate (Recombinant Laminin-511) (ReproCell) NutriStem® XF/FF™ medium (Biological Industries) for at least 15 passages prior to directed differentiation into HECs and hematopoietic lineages. HiPSCs were either passaged as cell clumps using Versene (Thermo Fisher) or single cells by Accutase or TripLE. To ensure genome stability of hiPSCs, G-banding karyotype analyses were routinely carried out at frequency of every 5 passages. Only hiPSCs with normal karyotypes were used in this study.

Recombinant protein BMP4 and oncostatin M (OSM) were purchased from Humanzyme. VEGF, bFGF, TPO, SCF, IL-3, IL-6, IL-9, IL-7, IL-15, sDLL-1 were purchased from Peprotech. EPO was purchased from eBioscience (Thermal Fisher). Small molecule Y27632 was purchased from Stemgent/Reprocell. CHIR99021 was purchased from TOCRIS Bioscience. Small molecule SB431542 was purchased from Reagent Direct. SR1 was purchased from StemCell Technologies.

Fluorochrome conjugated antibodies for flow cytometer analysis of CD31, CD144, CD34, CD43, CD235a, CD41a, CD42b, CD56, CD16, CD19, CD45, CD3, TCR, NKG2D, NKp44, NKp46 were purchased from BD Biosciences. CD133-APC and KIR2DS4-PE, KIR2DL1/DS1-PE and KIR3DL1/DS1-PE were purchased from Miltenyi. Oct-4 FITC was purchased from Cell Signaling. Unconjugated Mouse anti-human antibodies of CD31, CD34, CD43 were purchased from DAKO/Agilent.

Pre-Conditioning of hiPSCs for 3D Differentiation

HiPSC cells were cultured on a matrix such as Laminin 521 or Laminin 511 in NutriStem® hPSC XF medium (Biological Industries USA). Confluent and undifferentiated hiPSCs were passaged using Accutase (Innovative Cell Technologies, Inc) or TripLE (Thermo Fisher) and seeded onto a surface coated with reduced (½) concentration of matrix at density of 6-8×10⁴ cells per cm² in NutriStem® supplemented with 1 μM of Y27632 and culture for 3-7 days. HiPSCs were expanded in this condition for 3-5 passages. The undifferentiated status of hiPSCs is quantitated with the expression level of Oct-4 by flow cytometry analysis (over 95% Oct-4 positive). To initiate 3D suspension culture, confluent undifferentiated hiPSCs were dissociated by Accutase or TripLE and were seeded into a spinner flask at a density of 1×10⁶ cell/ml in NutriStem® supplemented with Y27632 (1 μM). The cells were cultured uninterrupted for 48 hours with agitation rate of 50-80 in a 30-ml spinner flask (Abel Biott). Forty-eight hours after seeding, a small sample was taken out, and the morphology and sphere sizes were examined Periodically media were refreshed until sphere sizes reached 250-300 micrometers in diameter. For passaging, hiPSC spheres were washed with PBS (Mg⁻, Ca⁻), and then dissociated by Accutase or TripLE. Dissociated hiPSC single cells were then seeded at a desired density for either expansion or initiation of hematopoietic differentiation.

Stepwise Induction of hiPSCs into HEC and Hematopoietic Lineages

This new 3D differentiation process was specifically developed to achieve the following 4 targets: (1) consistent and high efficiency generation of HEC population; (2) efficient transition from HEC intermediates to hematopoietic lineages; (3) maintenance of strong CD34⁺ population in long term culture; and (4) maximization to harvest high quality HPCs with all lineage specificities.

To determine optimal seeding density for efficient HEC differentiation, dissociated hiPSC suspensions were seeded at 3 different densities (0.67, 1, and 1.33×10⁶ cells/ml) in HEC induction medium M1 (NutriStem® supplemented with Y27632) for 12 hours. Average hiPSC sphere sizes were measured. Typical sphere sizes were between 80-150 micrometers in diameter depending on seeding densities. To initiate HE differentiation, NutriStem® with Y27632 was removed and replaced with the HEC induction medium M2 (growth factor-free NutriStem® hPSC XF Medium) supplemented with BMP4, VEGF, and bFGF at the concentration range of 25-50 ng/ml). HiPSC spheres in M2 were cultured under hypoxia condition (5% oxygen) for 4 days followed by 2 additional days in normal oxygen concentration of 20%. Media were changed daily, small molecule CHIR99021 was added at 3 μM for Day 3 and 4, and small molecule SB431542 was added at 3 μM at Day 4 and 5 (See FIG. 2). On Day 6 of HEC differentiation, cell spheres were dissociated into single cell suspension by treatment of TripLE for 15-30 mins at 37° C. The expression of HEC specific surface markers CD31, CD144 (VE-Cadherin), CD34, and CD43 was analyzed using flow cytometry. Successful HEC differentiation yields 30-70% CD31⁺ and CD144⁺ cells, as well as 15-30% CD34⁺ and 7.5-20% CD43⁺ cells. The HEC-containing spheres can be transitioned into hematopoietic commitment and expansion medium M3 (FIG. 2).

### Hematopoiefic Progenitors Release, Harvest, and Characterization

HEC is a bi-potent mesodermal intermediate cell population capable of becoming either endothelial or hematopoietic lineages. In order to maximize hematopoietic lineage output in our newly development platform, hematopoietic expansion medium M3 supplemented with TPO (10-25 ng/ml), SCF (10-25 ng/ml), Flt3L (10-25 ng/ml), IL-3 (2-10 ng/ml), IL-6 (2-10 ng/ml), SR1 (0.75 μM), OSM (2-10 ng/ml), and EPO (2 U/ml) was used for 5 days of phase 1 expansion. Hematopoietic differentiation/expansion medium M4 supplemented with TPO (10-25 ng/ml), SCF (10-25 ng/ml), Flt3L (10-25 ng/ml), IL-3 (2-10 ng/ml), IL-6 (2-10 ng/ml), SR1 (0.75 μM), OSM (2-10 ng/ml), and EPO (3 U/ml) was used in phase 2 expansion (up to 40 days). Media were changed daily and released progenitor cells were harvested from media by centrifugation and analyzed for surface lineage specific markers such as CD41 (megakaryocyte progenitors), CD235a (erythrocyte progenitors), CD34⁺CD45⁺ (early lymphoid/myeloid lineage progenitors), CD56⁺ (NK lineage progenitors), and CD34⁺CD133⁺ (hematopoietic stem cells).

### Morphological and Immunofluorescence Analysis of Stepwise Induction of HEC Population in 3D Cell Spheres

Starting from Day 0, undifferentiated hiPSC spheres, as well as differentiated spheres at various stages of processes, were collected and fixed in 4% paraformaldehyde in PBS at 4° C. for 1 hour. Spheres were then washed (once with PBS) and embedded with OCT at −20° C. for 1 hr. Frozen spheres were sectioned at 10-15 micrometers in thickness by a Leica CM1900 Cryostat. Sections were mounted onto positively charged glass slides and air dried for minimum of 1 hour at RT. Sphere sections were fixed again using freshly made cold (4° C.) 4% Paraformaldehyde (PFA) in PBS for 10 minutes, followed by 3 washes in PBS. For histological examination, slides were stained with hematoxylin solution for 30 sec, rinsed with tap water and mounted with an aqueous mount (Vector Lab). The morphologies of spheres were recorded by a color imaging system under the brightfield microscope.

For immunofluorescence staining, specimens were treated with blocking solution (DAKO/Agilent) for 30 mins at RT, followed by incubation with or without unconjugated primary antibodies (CD31, CD34, CD43, diluted with blocking solution at ratio of 1:50-100) at RT for 1 hour. Slides were washed with PBS 3 times and incubated with matching Alexa 488-conjugated donkey anti-mouse antibody (Thermo Fisher) diluted with blocking solution at 1:200 or 1:400 ratio for 1 hour at RT. Slides were washed with PBS 3 times again and mounted with mounting medium containing DAPi. Expression of HEC and/or hematopoietic markers on cell sphere sections were visualized by fluorescence microscopic imaging system (Nikon, Eclipse).

### Purification and Characterization of CD34⁺ Population

Cell spheres at various differentiation stages were collected and dissociated into single cells for CD34⁺ population enrichment. The dissociation of early spheres (up to Day 12) can be achieved by incubation with TripLE only for 15 mins to 1 hour at 37° C. For spheres after Day 12, a pre-incubation for 3-24 hours at 37° C. with collagenase IV (Thermo-Fisher) at the concentration of 1 mg/ml will be required in addition to TripLE dissociation thereafter. At the end of dissociation, the cell suspension was filtered through a strainer with 40 μm mesh to remove any large cell clumps. Specific cell population enrichment was performed using Miltenyi CD34 and CD45 microbead kit (Miltenyi). CD133+ and CD133⁻ HPC population were separated by CD133 microbeads kit (Miltenyi) following manufacturer's instruction. Cells of different fractions were analyzed by flow cytometry for CD34, CD45, and CD133 expression.

CD34⁺, CD34⁻CD45⁺, and CD34⁻CD45⁻ population purified from spheres at differentiation days were used for hematopoietic colony forming assay. Briefly, 2,000 cells from each of the three fractions were mixed with 1 ml Methcult H4436 (Stemcell Technologies) and seeded into 24-well ultralow attachment plates. The growth of colonies was monitored by microscope observation daily for up to 25 days. The morphology and quantity of hematopoietic colonies were recorded by photography and manual counting.

Megakaryocyte (MK) Lineage Specific Differentiation and Generation of Platelets from HPCs

HPCs released from Day 8 to Day 10 of differentiation were collected and cultured in vitro using conditions favoring the MK lineage as reported previously (Feng et al. 2014; Thon et al. 2014). StemSpan™-ACF (STEMCELL Technologies Inc.) medium was supplemented with TPO, SCF, IL-6 and IL-9 and heparin (5 U/ml) in ultralow attachment plates (Corning). Five micromolar Y-27632 was added for the first 3 days of culture, and cells were incubated in 7% CO₂ at 39° C. Cell densities were monitored daily and fresh medium was added to maintain 10⁶ cells/ml for the first 4 days. The maturation of MKs from MK progenitors (MKP) was monitored by analyzing CD41a and CD42b expression. Once proplatelet morphology (FIG. 12) was observed, platelets were collected for 3-5 consecutive days and analyzed for CD41a/CD42b expression.

### NK Lineage-Specific Differentiation of HPCs In Vitro

HPCs released at Day 8, Day 11, and Day 18 of differentiation were collected and cultured in vitro using conditions favoring NK lineage development as reported (Kaufman 2009; Knorr et al. 2013) with modifications. Two different basal media were used for comparison, supplemented with 10% FBS, SCF (10 ng/mL), Flt-3 (5 ng/mL), IL-7 (5 ng/mL), IL-15 (10 ng/mL), sDLL-1 (50 ng/mL), IL-6 (10 ng/mL), OSM (10 ng/mL), and Heparin (3 U/mL). All cells were cultured in ultralow attachment surface at a density of 2×10⁶ cells/ml. Media were changed every other day, and expression of NK lineage marker CD56 was monitored for up to 25 days. For NK lineage development using cellular scaffolds, between 2-4×10⁶ HECs harvested from Day 6 spheres were loaded into a Cell-Mate 3D μGel 40 kit (BRTI Life Sciences) according to manufacturer's instructions. The loaded scaffolds were cultured in suspension in the serum-free version of NK promoting medium supplemented with IL-3 (2-10 ng, for the first 5 days only), IL-7 (5-20 ng/ml), IL-15 (5-20 ng/ml), SCF (10-100 ng/ml), Flt3L (10-100 ng/ml), sDLL-1 (20-100 ng/ml), and Heparin. Media were changed every other day. Cells released from the scaffolds in suspension were monitored for NK specific markers CD56, NKp44, NKp46, NKG2D, KIRs, TCR, CD3, and CD19 for up to 50 days.

Cytotoxic of Human iPS-Derived NK Cells on K562 Erythroleukemia Cells

Reagent kits for quantitative determination of the cytotoxic activity of NK cells were purchased from Glycotope Biotechnology GmbH (Heidelberg, Germany). Briefly, target cells (T) K562 GFP cells were thawed and cell viability were measured (>92%). Adjust the K562 concentration to 1×10⁵ cells/ml with complete medium (provided). Harvest iPS-NK from NK culture was used directly as effector cells (E) without purification. Adjust Effector cell concentration to 5×10⁶/ml with complete medium. In 12×75 mm culture tubes, effector cells with or without IL-2 (200 U/ml) were mixed with Target cells at T:E ratio of 1:50, 1:25 and 1:12.5 respectively and K562 cell only was used as control. Vortex all tubes, centrifuge tubes for 2-3 min at 120 g. Incubate the tubes for 120 mins in CO₂ incubator. Add 50 ml DNA staining solution to each tube, vortex and incubate 5 min on ice. Measure the cell suspension within 30 min after addition of DNA staining solution with flow channel of GFP and PE.

### EQUIVALENTS

The present disclosure provides among other things in vitro cell culture systems and use thereof. While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### INCORPORATION BY REFERENCE

All publications and patents mentioned herein are hereby incorporated by reference in their entirety as if each individual publication or patent was specifically and individually indicated to be incorporated by reference.

### REFERENCES

Addison, E. G., J. North, I. Bakhsh, C. Marden, S. Haq, S. Al-Sarraj, R. Malayeri, R. G. Wickremasinghe, J. K. Davies, and M. W. Lowdell. 2005. ‘Ligation of CD8alpha on human natural killer cells prevents activation-induced apoptosis and enhances cytolytic activity’, Immunology, 116: 354-61.
Ahmad, F., H. S. Hong, M. Jackel, A. Jablonka, I. N. Lu, N. Bhatnagar, J. M. Eberhard, B. A. Bollmann, M. Ballmaier, M. Zielinska-Skowronek, R. E. Schmidt, and D. Meyer-Olson. 2014. ‘High frequencies of polyfunctional CD8+NK cells in chronic HIV-1 infection are associated with slower disease progression’, J Virol, 88: 12397-408.
Algarra, I., A. Garcia-Lora, T. Cabrera, F. Ruiz-Cabello, and F. Garrido. 2004. ‘The selection of tumor variants with altered expression of classical and nonclassical MHC class I molecules: implications for tumor immune escape’, Cancer Immunol Immunother, 53: 904-10.
Bachanova, V., S. Cooley, T. E. Defor, M. R. Verneris, B. Zhang, D. H. McKenna, J. Curtsinger, A. Panoskaltsis-Mortari, D. Lewis, K. Hippen, P. McGlave, D. J. Weisdorf, B. R. Blazar, and J. S. Miller. 2014. ‘Clearance of acute myeloid leukemia by haploidentical natural killer cells is improved using IL-2 diphtheria toxin fusion protein’, Blood, 123: 3855-63.
Bashirova, A. A., M. P. Martin, D. W. McVicar, and M. Carrington. 2006. ‘The killer immunoglobulin-like receptor gene cluster: tuning the genome for defense’, Annu Rev Genomics Hum Genet, 7: 277-300.
Burns, L. J., D. J. Weisdorf, T. E. DeFor, D. H. Vesole, T. L. Repka, B. R. Blazar, S. R. Burger, A. Panoskaltsis-Mortari, C. A. Keever-Taylor, M. J. Zhang, and J. S. Miller. 2003. ‘IL-2-based immunotherapy after autologous transplantation for lymphoma and breast cancer induces immune activation and cytokine release: a phase I/II trial’, Bone Marrow Transplant, 32: 177-86.
Choi, K. D., M. Vodyanik, and Slukvin, II. 2008. ‘Hematopoietic differentiation.’ in, StemBook (Harvard Stem Cell Institute
Copyright: (c) 2012 Kyung-Dal Choi, Maxim Vodyanik, and Igor I Slukvin: Cambridge (Mass.)).
Costello, R. T., J. A. Gastaut, and D. Olive. 1999. ‘Tumor escape from immune surveillance’, Arch Immunol Ther Exp (Warsz), 47: 83-8.
Cunha, B., T. Aguiar, M. M. Silva, R. J. Silva, M. F. Sousa, E. Pineda, C. Peixoto, M. J. Carrondo, M. Serra, and P. M. Alves. 2015. ‘Exploring continuous and integrated strategies for the up- and downstream processing of human mesenchymal stem cells’, J Biotechnol, 213: 97-108.
D'Souza, S. S., J. Maufort, A. Kumar, J. Zhang, K. Smuga-Otto, J. A. Thomson, and Slukvin, II. 2016. ‘GSK3beta Inhibition Promotes Efficient Myeloid and Lymphoid Hematopoiesis from Non-human Primate-Induced Pluripotent Stem Cells’, Stem Cell Reports, 6: 243-56.
Daley, G. Q. 2003. ‘From embryos to embryoid bodies: generating blood from embryonic stem cells’, Ann NY Acad Sci, 996: 122-31.
de Pooter, R., and J. C. Zuniga-Pflucker. 2007. ‘T-cell potential and development in vitro: the OP9-DL1 approach’, Curr Opin Immunol, 19: 163-8.
deMagalhaes-Silverman, M., A. Donnenberg, B. Lembersky, E. Elder, J. Lister, W. Rybka, T. Whiteside, and E. Ball. 2000. ‘Posttransplant adoptive immunotherapy with activated natural killer cells in patients with metastatic breast cancer’, J Immunother, 23: 154-60.
Ditadi, A., C. M. Sturgeon, J. Tober, G. Awong, M. Kennedy, A. D. Yzaguirre, L. Azzola, E. S. Ng, E. G. Stanley, D. L. French, X. Cheng, P. Gadue, N. A. Speck, A. G. Elefanty, and G. Keller. 2015. ‘Human definitive haemogenic endothelium and arterial vascular endothelium represent distinct lineages’, Nat Cell Biol, 17: 580-91.
Drake, A. C., M. Khoury, I. Leskov, B. P. Iliopoulou, M. Fragoso, H. Lodish, and J. Chen. 2011. ‘Human CD34+CD133+ hematopoietic stem cells cultured with growth factors including Angptl5 efficiently engraft adult NOD-SCID Il2rgamma−/−(NSG) mice’, PLoS One, 6: e18382.
Ebina, W., and D. J. Rossi. 2015. ‘Transcription factor-mediated reprogramming toward hematopoietic stem cells’, Embo j, 34: 694-709.
Feng, Q., N. Shabrani, J. N. Thon, H. Huo, A. Thiel, K. R. Machlus, K. Kim, J. Brooks, F. Li, C. Luo, E. A. Kimbrel, J. Wang, K. S. Kim, J. Italiano, J. Cho, S. J. Lu, and R. Lanza. 2014. ‘Scalable generation of universal platelets from human induced pluripotent stem cells’, Stem Cell Reports, 3: 817-31.
Grupp, S. A., M. Kalos, D. Barrett, R. Aplenc, D. L. Porter, S. R. Rheingold, D. T. Teachey, A. Chew, B. Hauck, J. F. Wright, M. C. Milone, B. L. Levine, and C. H. June. 2013. ‘Chimeric antigen receptor-modified T cells for acute lymphoid leukemia’, N Engl J Med, 368: 1509-18.
Houchins, J. P., T. Yabe, C. McSherry, and F. H. Bach. 1991. ‘DNA sequence analysis of NKG2, a family of related cDNA clones encoding type II integral membrane proteins on human natural killer cells’, J Exp Med, 173: 1017-20.
Ivanovs, A., S. Rybtsov, E. S. Ng, E. G. Stanley, A. G. Elefanty, and A. Medvinsky. 2017. ‘Human haematopoietic stem cell development: from the embryo to the dish’, Development, 144: 2323-37.
Kaufman, D. S. 2009. ‘Toward clinical therapies using hematopoietic cells derived from human pluripotent stem cells’, Blood, 114: 3513-23.
Knorr, D. A., Z. Ni, D. Hermanson, M. K. Hexum, L. Bendzick, L. J. Cooper, D. A. Lee, and D. S. Kaufman. 2013. ‘Clinical-scale derivation of natural killer cells from human pluripotent stem cells for cancer therapy’, Stem Cells Transl Med, 2: 274-83.
Kochenderfer, J. N., W. H. Wilson, J. E. Janik, M. E. Dudley, M. Stetler-Stevenson, S. A. Feldman, I. Maric, M. Raffeld, D. A. Nathan, B. J. Lanier, R. A. Morgan, and S. A. Rosenberg. 2010. ‘Eradication of B-lineage cells and regression of lymphoma in a patient treated with autologous T cells genetically engineered to recognize CD19’, Blood, 116: 4099-102.
Lanier, L. L. 2001. ‘Face off—the interplay between activating and inhibitory immune receptors’, Curr Opin Immunol, 13: 326-31.
Li, Y., D. L. Hermanson, B. S. Moriarity, and D. S. Kaufman. 2018. ‘Human iPSC-Derived Natural Killer Cells Engineered with Chimeric Antigen Receptors Enhance Anti-tumor Activity’, Cell Stem Cell, 23: 181-92 e5.
Lu, S. J., Q. Feng, S. Caballero, Y. Chen, M. A. Moore, M. B. Grant, and R. Lanza. 2007. ‘Generation of functional hemangioblasts from human embryonic stem cells’, Nat Methods, 4: 501-9.
Lu, S. J., Q. Feng, J. S. Park, L. Vida, B. S. Lee, M. Strausbauch, P. J. Wettstein, G. R. Honig, and R. Lanza. 2008. ‘Biologic properties and enucleation of red blood cells from human embryonic stem cells’, Blood, 112: 4475-84.
Lu, S. J., F. Li, H. Yin, Q. Feng, E. A. Kimbrel, E. Hahm, J. N. Thon, W. Wang, J. E. Italiano, J. Cho, and R. Lanza 2011. ‘Platelets generated from human embryonic stem cells are functional in vitro and in the microcirculation of living mice’, Cell Res, 21: 530-45.
MacLeod, R. A., S. Nagel, M. Kaufmann, K. Greulich-Bode, and H. G. Drexler. 2002. ‘Multicolor-FISH analysis of a natural killer cell line (NK-92)’, Leak Res, 26: 1027-33.
McCall, M., and A. M. Shapiro. 2012. ‘Update on islet transplantation’, Cold Spring Harb Perspect Med, 2: a007823.
Mehta, R. S., and K. Rezvani. 2018. ‘Chimeric Antigen Receptor Expressing Natural Killer Cells for the Immunotherapy of Cancer’, Front Immunol, 9: 283.
Mohtashami, M., and J. C. Zuniga-Pflucker. 2006. ‘Three-dimensional architecture of the thymus is required to maintain delta-like expression necessary for inducing T cell development’, J Immunol, 176: 730-4.
Ng, E. S., R. P. Davis, T. Hatzistavrou, E. G. Stanley, and A. G. Elefanty. 2008. ‘Directed differentiation of human embryonic stem cells as spin embryoid bodies and a description of the hematopoietic blast colony forming assay’, Curr Protoc Stem Cell Biol, Chapter 1: Unit 1D.3.
Palis, J. 2016. ‘Hematopoietic stem cell-independent hematopoiesis: emergence of erythroid, megakaryocyte, and myeloid potential in the mammalian embryo’, FEBS Lett, 590: 3965-74.
Racle, J., K. de Jonge, P. Baumgaertner, D. E. Speiser, and D. Gfeller. 2017. ‘Simultaneous enumeration of cancer and immune cell types from bulk tumor gene expression data’, Elife, 6.
Ruggeri, L., M. Capanni, E. Urbani, K. Perruccio, W. D. Shlomchik, A. Tosti, S. Posati, D. Rogaia, F. Frassoni, F. Aversa, M. F. Martelli, and A. Velardi. 2002. ‘Effectiveness of donor natural killer cell alloreactivity in mismatched hematopoietic transplants’, Science, 295: 2097-100.
Rutjens, E., S. Mazza, R. Biassoni, G. Koopman, E. Ugolotti, M. Fogli, R. Dubbes, P. Costa, M. C. Mingari, E. J. Greenwood, L. Moretta, A. De Maria, and J. L. Heeney. 2010. ‘CD8+NK cells are predominant in chimpanzees, characterized by high NCR expression and cytokine production, and preserved in chronic HIV-1 infection’, Eur J Immunol, 40: 1440-50.
Salvagiotto, G., S. Burton, C. A. Daigh, D. Rajesh, Slukvin, II, and N. J. Seay. 2011. ‘A defined, feeder-free, serum-free system to generate in vitro hematopoietic progenitors and differentiated blood cells from hESCs and hiPSCs’, PLoS One, 6: e17829.
Schonfeld, K., C. Sahm, C. Zhang, S. Naundorf, C. Brendel, M. Odendahl, P. Nowakowska, H. Bonig, U. Kohl, S. Kloess, S. Kohler, H. Holtgreve-Grez, A. Jauch, M. Schmidt, R. Schubert, K. Kuhlcke, E. Seifried, H. G. Klingemann, M. A. Rieger, T. Tonn, M. Grez, and W. S. Wels. 2015. ‘Selective inhibition of tumor growth by clonal NK cells expressing an ErbB2/HER2-specific chimeric antigen receptor’, Mol Ther, 23: 330-8.
Shah, N., B. Martin-Antonio, H. Yang, S. Ku, D. A. Lee, L. J. Cooper, W. K. Decker, S. Li, S. N. Robinson, T. Sekine, S. Parmar, J. Gribben, M. Wang, K. Rezvani, E. Yvon, A. Najjar, J. Burks, I. Kaur, R. E. Champlin, C. M. Bollard, and E. J. Shpall. 2013. ‘Antigen presenting cell-mediated expansion of human umbilical cord blood yields log-scale expansion of natural killer cells with anti-myeloma activity’, PLoS One, 8: e76781.
Shapiro, A. M., C. Ricordi, B. J. Hering, H. Auchincloss, R. Lindblad, R. P. Robertson, A. Secchi, M. D. Brendel, T. Berney, D. C. Brennan, E. Cagliero, R. Alejandro, E. A. Ryan, B. DiMercurio, P. Morel, K. S. Polonsky, J. A. Reems, R. G. Bretzel, F. Bertuzzi, T. Froud, R. Kandaswamy, D. E. Sutherland, G. Eisenbarth, M. Segal, J. Preiksaitis, G. S. Korbutt, F. B. Barton, L. Viviano, V. Seyfert-Margolis, J. Bluestone, and J. R. Lakey. 2006. ‘International trial of the Edmonton protocol for islet transplantation’, N Engl J Med, 355: 1318-30.
Sivori, S., M. Vitale, L. Morelli, L. Sanseverino, R. Augugliaro, C. Bottino, L. Moretta, and A. Moretta. 1997. ‘p46, a novel natural killer cell-specific surface molecule that mediates cell activation’, J Exp Med, 186: 1129-36.
Sturgeon, C. M., A. Ditadi, G. Awong, M. Kennedy, and G. Keller. 2014. ‘Wnt signaling controls the specification of definitive and primitive hematopoiesis from human pluripotent stem cells’, Nat Biotechnol, 32: 554-61.
Sugimura, R., D. K. Jha, A. Han, C. Soria-Valles, E. L. da Rocha, Y. F. Lu, J. A. Goettel, E. Serrao, R. G. Rowe, M. Malleshaiah, I Wong, P. Sousa, T. N. Zhu, A. Ditadi, G. Keller, A. N. Engelman, S. B. Snapper, S. Doulatov, and G. Q. Daley. 2017. ‘Haematopoietic stem and progenitor cells from human pluripotent stem cells’, Nature, 545: 432-38.
Swiers, G., C. Rode, E. Azzoni, and M. F. de Bruijn. 2013. ‘A short history of hemogenic endothelium’, Blood Cells Mol Dis, 51: 206-12.
Thon, J. N., L. Mazutis, S. Wu, J. L. Sylman, A. Ehrlicher, K. R. Machlus, Q. Feng, S. Lu, R. Lanza, K. B. Neeves, D. A. Weitz, and J. E. Italiano, Jr. 2014. ‘Platelet bioreactor-on-a-chip’, Blood, 124: 1857-67.
Vitale, M., C. Bottino, S. Sivori, L. Sanseverino, R. Castriconi, E. Marcenaro, R. Augugliaro, L. Moretta, and A. Moretta. 1998. ‘NKp44, a novel triggering surface molecule specifically expressed by activated natural killer cells, is involved in non-major histocompatibility complex-restricted tumor cell lysis’, J Exp Med, 187: 2065-72.
Wahlster, L., and G. Q. Daley. 2016. ‘Progress towards generation of human haematopoietic stem cells’, Nat Cell Biol, 18: 1111-17.
Watarai, H., A. Rybouchkin, N. Hongo, Y. Nagata, S. Sakata, E. Sekine, N. Dashtsoodol, T. Tashiro, S. Fujii, K. Shimizu, K. Mori, K. Masuda, H. Kawamoto, H. Koseki, and M. Taniguchi. 2010. ‘Generation of functional NKT cells in vitro from embryonic stem cells bearing rearranged invariant Valpha14-Jalpha18 TCRalpha gene’, Blood, 115: 230-7.
Winters, J. L. 2006. ‘Complications of donor apheresis’, J Clin Apher, 21: 132-41.
Yawata, M., N. Yawata, L. Abi-Rached, and P. Parham. 2002. ‘Variation within the human killer cell immunoglobulin-like receptor (KIR) gene family’, Crit Rev Immunol, 22: 463-82.
Ye Li, David L. Hermanson, Branden S. Moriarity, and Dan S. Kaufman. 2018 Human iPSC-derived natural killer cells engineered with chimeric antigen receptors enhance anti-tumor activity. Cell Stem Cell, 23: 181-192.
Yokoyama, W. M. 1998. ‘Natural killer cell receptors’, Curr Opin Immunol, 10: 298-305.
Yuan, S., A. Ziman, B Smeltzer, Q. Lu, and D. Goldfinger. 2010. ‘Moderate and severe adverse events associated with apheresis donations: incidences and risk factors’, Transfusion, 50: 478-86.
Zeng, J., S. Y. Tang, L. L. Toh, and S. Wang. 2017. ‘Generation of “Off-the-Shelf” Natural Killer Cells from Peripheral Blood Cell-Derived Induced Pluripotent Stem Cells’, Stem Cell Reports, 9: 1796-812.

1. A method for in vitro production of hematopoietic lineage cells, comprising:
(a) providing a plurality of first spheres comprising pluripotent stem cells (PSCs) in a first culture medium, wherein the first spheres have an average size of about 60-150 micrometers, about 70-120 micrometers or about 80-100 micrometers in diameter; wherein preferably the first spheres are generated from 3-dimensional (3D) sphere culturing while monitoring sphere size;
(b) 3D sphere culturing the plurality of first spheres in a second culture medium to induce differentiation of the PSCs to generate a plurality of second spheres comprising hemogenic endothelial cells (HECs);
(c) 3D sphere culturing the plurality of second spheres in a third culture medium to induce differentiation of the HECs to generate a plurality of third spheres comprising hematopoietic progenitor cells (HPCs);
(d) permitting the HPCs to release from the plurality of third spheres to obtain a suspension of substantially single cells of HPCs; and
(e) optionally, further differentiating the suspension of substantially single cells of HPCs into common erythroid/megakaryocytic progenitor cells, erythrocytes, megakaryocytes, platelets, common lymphoid progenitor cells, lymphoid lineage cells, lymphocytes (such as T lymphocytes), natural killer (NK) cells, common myeloid progenitor cells, common granulomonocytic progenitor cells, monocytes, macrophages, and/or dendritic cells.

2. A method for in vitro production of lymphoid lineage cells, comprising:
(a) providing a plurality of first spheres comprising pluripotent stem cells (PSCs) in a first culture medium, wherein the first spheres have an average size of about 60-150 micrometers, about 70-120 micrometers or about 80-100 micrometers in diameter; wherein preferably the first spheres are generated from 3-dimensional (3D) sphere culturing while monitoring sphere size;
(b) 3D sphere culturing the plurality of first spheres in a second culture medium to induce differentiation of the PSCs to generate a plurality of second spheres containing hemogenic endothelial cells (HECs);
(c) enzymatically disassociating the plurality of second spheres to obtain a suspension of substantially single cells of HECs;
(d) seeding the substantially single cells of HECs into a scaffold that mimics in vivo hematopoietic niche; and
(e) culturing and differentiating, in the scaffold, the HECs into lymphoid lineage cells.

3. A method for in vitro production of lymphoid lineage cells, comprising:
(a) providing a plurality of first spheres comprising pluripotent stem cells (PSCs) in a first culture medium, wherein the first spheres have an average size of about 60-150 micrometers, about 70-120 micrometers or about 80-100 micrometers in diameter; wherein preferably the first spheres are generated from 3-dimensional (3D) sphere culturing while monitoring sphere size;
(b) 3D sphere culturing the plurality of first spheres in a second culture medium to induce differentiation of the PSCs to generate a plurality of second spheres containing hemogenic endothelial cells (HECs); and
(c) culturing and differentiating, in a scaffold-free third culture medium, the HECs in the second spheres into lymphoid lineage cells, while permitting the lymphoid lineage cells to release from the second spheres.

4. The method of claim 1, wherein the PCSs are embryonic stem cells or induced pluripotent stem cells, preferably from human.

5. The method of claim 1, wherein the PCSs are at least 95% positive for Oct-4 expression.

6. The method of claim 1, wherein each 3D sphere culturing step comprises culturing in a spinner flask or stir-tank bioreactor, preferably under continuous agitation.

7. The method of claim 1, wherein the first culture medium is a PSC culture medium supplemented with TGF-β of about 1-10 ng/mL, bFGF of about 10-500 ng/mL, and Y27632 of about 1-5 μM.

8. The method of claim 7, wherein the PSC culture medium is NutriStem®, mTeSR™1, mTeSR™2, TeSR™-E8™ or other culture medium suitable for 3D suspension culture.

9. The method of claim 1, wherein the second culture medium is a PSC culture medium supplemented with BMP4, VEGF and bFGF, each preferably at a concentration of about 25 to about 50 ng/mL, and optionally supplemented with CHIR99012 and/or SB431542, each preferably at a concentration of about 1-10, about 2-5, or about 3 μM.

10. The method of claim 9, wherein the PSC culture medium is NutriStem®, mTeSR™1, mTeSR™2, TeSR™-E8™ or other culture medium suitable for 3D suspension culture.

11. The method of claim 9, wherein the second culture medium is supplemented with (i) BMP4, VEGF and bFGF for a first period of time (e.g., day 1 and day 2), (ii) BMP4, VEGF, bFGF and CHIR99012 for a second period of time (e.g., day 3), (iii) BMP4, VEGF, bFGF, CHIR99012 and SB431542 for a third period of time (e.g., day 4), (iv) BMP4, VEGF, bFGF, and SB431542 for a fourth period of time (e.g., day 5), and (v) BMP4, VEGF and bFGF for a fifth period of time (e.g., day 6).

12. The method of claim 9, wherein said culturing in the second culture medium is under hypoxia condition (about 5% oxygen) for the first period of time through the third period of time (e.g., day 1 through day 4), followed by normal oxygen concentration of about 20% for the fourth period of time and the fifth period of time (e.g., day 5 and day 6).

13. The method of claim 1, wherein the third culture medium is a hematopoietic basal medium supplemented with one or more of TPO, SCF, Flt3L, IL-3, IL-6, IL-7, IL-15, SR1, sDLL-1, OSM and/or EPO.

14. The method of claim 13, wherein the hematopoietic basal medium is StemSpan™-ACF, PRIME-XV®, PromoCell® Hematopoietic Progenitor Expansion medium DXF and other culture system suitable for hematopoietic stem cell expansion.

15. The method of claim 1, wherein step (e) comprises culturing in a hematopoietic basal medium supplemented with one or more of TPO, SCF, Flt3L, IL-3, IL-6, IL-7, IL-15, SR1, sDLL-1, OSM and/or EPO.

16. The method of claim 15, wherein the hematopoietic basal medium is StemSpan™-ACF, PRIME-XV®, PromoCell® Hematopoietic Progenitor Expansion medium DXF and other culture medium suitable for lineage-specific expansion and maturation.

17. The method of claim 2, wherein the lymphoid lineage cells are T-cells, NK cells, dendritic cells and/or macrophages.

18. A composition for adoptive cell therapy, comprising a plurality of cells produced using the method of claim 1, wherein preferably the cells have been engineered to express a chimeric antigen receptor, a T-cell receptor or other receptor for disease antigens for the treatment of cancer or other immune diseases, wherein more preferably the cells are T-cells, NK cells, dendritic cells and/or macrophages.

19. Cells produced using the method of claim 1 for the treatment of cancer or other immune diseases, wherein preferably the cells have been engineered to express a chimeric antigen receptor, a T-cell receptor or other receptor for disease antigens, wherein more preferably the cells are T-cells, NK cells, dendritic cells and/or macrophages.
